(19) United States
(12) Patent Application Publication
(10) Pub. No.: US 2021/0071129 A1
(43) Pub. Date: Mar. 11, 2021

(54) Cell Culture Incubator System

(71) Applicant: Scientific Industries, Inc., Bohemia, NY (US)

(72) Inventor: Joseph G. Cremonese, Greensburg, PA (US)

(21) Appl. No.: 16/948,154

(22) Filed: Sep. 4, 2020

Related U.S. Application Data
(60) Provisional application No. 62/896,211, filed on Sep. 5, 2019.

(51) Int. Cl. C12M 1/00; G01N 21/64; C12M 1/34; C12M 3/06

(52) U.S. Cl. CPC C12M 41/14; G01N 21/64; C12M 41/26; C12M 27/16; C12M 41/40; C12M 41/06; C12M 41/34

(57) ABSTRACT

Disclosed is a cell culture incubator system having a sensor strip configured to be placed within a reaction vessel (e.g., incubator vessel) so as to position the sensor within media covering the cells. A reader is placed outside but adjacent the vessel to read the sensor so as to detect changes in dissolved O₂ and pH. The system is used to determine if the incubator environment has too much CO₂ and is therefore trending towards hypoxia and/or acidity, or has too much dissolved O₂ and is therefore trending toward oxygen toxicity via the cell nutrient media. In some embodiments, the system includes a rocker unit configured to rock the reaction vessel to enhance cell growth. In some embodiments, the system includes a gas circulation system to adjust the CO₂ and dissolved O₂ levels in the reaction vessel.

### CROSS-REFERENCE TO RELATED APPLICATIONS

This application is a non-provisional utility application which is related to and claims the benefit of U.S. Provisional Application No. 62/896,211 filed on Sep. 5, 2019, the entire contents of each being incorporated herein by reference.

### BACKGROUND OF THE INVENTION

### Field of the Invention

Disclosed is a cell culture incubator system having a sensor strip configured to be placed within a reaction vessel (e.g., incubator vessel) so as to position the sensor within media covering the cells. A reader is placed outside but adjacent to the vessel to read the sensor so as to detect changes in dissolved O₂ and pH. The system is used to determine if the incubator environment has too much CO₂ and is therefore trending towards hypoxia and/or acidity, or has too much dissolved O₂ and is therefore trending toward oxygen toxicity via the cell nutrient media. In some embodiments, the system includes a rocker unit configured to rock the reaction vessel to enhance cell growth. In some embodiments, the system includes a gas circulation system to adjust the CO₂ and dissolved O₂ levels in the reaction vessel.

### Background of the Related Art

Measuring dissolved oxygen (“DO”) and pH can provide a reliable approach to monitoring cell cultures. For instance, the biomass of growing cells can cause an increase in lactic acid within the cell growth media (e.g., nutrient media), which can be due to an end-product of cell metabolism. An increase in lactic acid can cause a steady decrease of pH level in the cell growth media, and the presence of DO can be an indicator of the health of the cell culture environment. Generally, optimal levels of DO in the cell growth media provide a healthy environment for cell growth. Thus, pH levels and DO levels can be indicators of conditions within the cell culture. For example, DO saturation with a positive rate of change can be indicative of equilibrium, whereas decreasing level of pH below a certain amount or at a certain rate can be indicative of over-growth in the cell culture (i.e., may require a subculture). Other conditions that can be determined may include, but are not limited to, an onset of programmed cell death (e.g., apoptosis), growth toward equilibrium, deviations from equilibrium, etc.

Changes in DO levels and pH levels can be monitored by placing a substrate within the cell growth media, the substrate being configured to generate light via fluorescence techniques. For example, a fluorescence-based patch (e.g., a sensor patch) containing fluorescent dye immobilized in a matrix can be placed within a reaction vessel containing the culture sample. Elicitation of fluorescence from chemicals within the patch can be performed, where emitted light due to induced fluorescence can be monitored via a reader (e.g., electro-optics and/or other system components) located outside the reaction vessel. Thus, electro-optical measurements can be conducted on the cell culture in a minimally invasive and/or non-invasive manner. Furthermore, the patch can be structured such that emitted light due to the fluorescence can occur at different wavelengths, the wavelengths being a function of changes in DO levels and/or pH levels occurring within a growth media.

Conventional cell culture incubator systems can be appreciated from U.S. Pat. No. 4,839,292; US 2008/0024779; US 2011/0188053; RA.GUPATHY, V. et al., Non-Invasive Optical Sensor Based Approaches for Monitoring Virus Culture to Minimize BSL3 Laboratory Entry, Sensors, 2015, 14864-14870, Vol 15, MDPI, Basel, Switzerland; GE, X. et al., Low-Cost Noninvasive Optical CO₂ Sensing System for Fermentation and Cell Culture, Biotechnology and BioEngineering, 2005, 329-334, Vol 89, No. 3, Wiley Periodicals, Inc.; GE, X. et al., Validation of An Optical Sensor-Based High-Throughput Bioreactor System for Mammalian Cell Culture, Journal of Biotechnology, 2006, 291-306, Vol. 122, Elsevier.com; RAO, G et al., Disposable Bioprocessing: The Future Has Arrived, Biotechnology and BioEngineering, 2009, 348-356, Vol 102, No. 2, Wiley Periodicals, Inc.; HANSON, M. A., Comparisons of Optical pH and Dissolved Oxygen Sensors with Traditional Electrochemical Probes During Mammalian Cell Culture, 2007, 833-841, Vol. 4, No. 4, Wiley Periodicals, Inc.; and Schiefelbein, et. al “Oxygen supply in disposable shake-flasks: prediction of oxygen transfer rate, oxygen saturation and maximum cell concentration during aerobic growth”, Springer Science+Business Media. Jan. 10, 2013. Conventional systems are limited in that they generally require a reaction vessel to include a sensor patch already secured or attached to the reaction vessel, which in turn may require the sensor patch to be sterilized before use. In addition, such a set up requires proper alignment between the sensor patch and the reader. Conventional systems are further limited in that they fail to provide any means to effectively perturb the media within which the cells are grown. Perturbing the media, however, can enhance growth of the cells. Conventional systems are further limited in that they fail to provide an adequate means to adjust the DO and CO₂ levels if they are detected to be at undesired levels.

### BRIEF SUMMARY OF THE INVENTION

Embodiments can relate to a cell culture incubator system having a sensor strip configured to be placed within a reaction vessel. The sensor strip can be configured so that when inserted, the sensor is positioned within media covering the cells. A reader is placed outside but adjacent to the vessel to read the sensor so as to detect changes in dissolved O₂ and pH. The system is used to determine if the incubator environment has too much CO₂ and is therefore trending towards hypoxia and/or acidity, or has too much dissolved O₂ and is therefore trending toward oxygen toxicity via the cell nutrient media. Other conditions that can be determined may include the onset of apoptosis, growth toward equilibrium, deviations from equilibrium, etc.

In some embodiments, the system includes a gas circulation system to adjust the CO₂ and dissolved O₂ levels in the reaction vessel.

Some embodiments can include an incubator system in which a plurality of reaction vessels is being monitored. The incubator system can be a controlled housing configured to adjust lighting, temperature, pressure, etc. for the plurality of reaction vessels. The incubator system can house the plurality of reaction vessels so that each reaction vessel is in fluid communication with the gas circulation system. The gas circulation system can adjust the CO₂ and dissolved O₂ levels of each individual reaction vessel separately or in unison.

In some embodiments, the system includes a rocker unit configured to rock the reaction vessel to enhance cell growth.

In some embodiments, the system includes a rotation unit configured to rotate the reaction vessel to further enhance cell growth.

In some embodiments, the system can be part of or connected to a computer device or a computer system. With such embodiments, a feedback loop can be created between the reader, the gas circulation system, the incubator housing, and/or the rocker to monitor the CO₂ and dissolved O₂ levels in real-time and to adjust the CO₂ and dissolved O₂ levels, the rocking, the rotation, the temperature, the lighting, the pressure, etc. in response thereto.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present invention will be more apparent from the following more particular description thereof, presented in conjunction with the following drawings, in which:

FIG. 1 shows an exemplary L-shaped sensor strip.

FIG. 2 shows an embodiment of the L-shaped sensor strip within a vertically arranged reaction vessel.

FIG. 3 shows an exemplary angled-shaped sensor strip.

FIG. 4 shows an embodiment of the angled-shaped sensor strip within a horizontally arranged reaction vessel.

FIG. 5 shows an exemplary multi-welled reaction vessel.

FIG. 6 shows an exemplary reader unit.

FIG. 7 shows an embodiment of the reader unit juxtaposed with an embodiment of the vertically aligned reaction vessel.

FIG. 8 shows an embodiment of the reader unit placed underneath an embodiment of the vertically aligned reaction vessel.

FIG. 9 shows an embodiment of the reader unit placed underneath an embodiment of the horizontally aligned reaction vessel.

FIG. 10 shows an exemplary incubation system having a rocker unit and/or a rotary unit.

FIG. 11 illustrates exemplary motions that can be imparted onto a reaction vessel due to embodiments of the rocker and/or rotary units.

FIG. 12 shows an exemplary gas circulation system.

FIG. 13 shows an exemplary reaction vessel having a gas inlet/outlet.

FIG. 14 shows a block diagram of an exemplary reader.

FIG. 15 shows an exemplary block diagram of an embodiment of the reader in connection with a computer device.

FIGS. 16-18 are various views of an exemplary beam combiner assembly with a single detector that may be used with an embodiment of the reader.

FIGS. 19-21 are various views of an exemplary beam combiner assembly with a dual detector that may be used with an embodiment of the reader.

FIGS. 22-24 are various views of an exemplary circuit board block with a single detector that may be used with an embodiment of the reader.

FIGS. 25-27 are various views of an exemplary circuit board block with a dual detector that may be used with an embodiment of the reader.

FIGS. 28-29 are side cross-sectional views of embodiments of beam combiner assemblies showing a non-shifted filter/mirror arrangement and a shifted filter/mirror arrangement, respectively, that may be used with an embodiment of the reader.

FIGS. 30-31 shows variations in height between an embodiment of a circuit board block with a non-cylindrical filter and non-shifted mirror, and an embodiment of a circuit board block with a cylindrical filter and a shifted mirror, respectively, that may be used with an embodiment of the reader.

FIGS. 32-38 show various views of an embodiment of a beam combiner assembly with an exemplary offsetting generator that may be used with an embodiment of the reader.

FIG. 39 is a side view of an exemplary beam combiner assembly with a widened beam combiner window that may be used with an embodiment of the reader.

FIGS. 40 and 41 are an exemplary user interfaces that may be displayed on a computer device that can be used with an embodiment of the reader.

FIGS. 42A-42B show an exemplary method of using an embodiment of the reader. Note that FIG. 42B is a continuation of the process that begins on FIG. 42A.

FIG. 43 is an exemplary computer system that may be used with the reader.

### DETAILED DESCRIPTION OF THE INVENTION

The following description is of an embodiment presently contemplated for carrying out the present invention. This description is not to be taken in a limiting sense, but is made merely for the purpose of describing the general principles and features of the present invention. The scope of the present invention should be determined with reference to the claims.

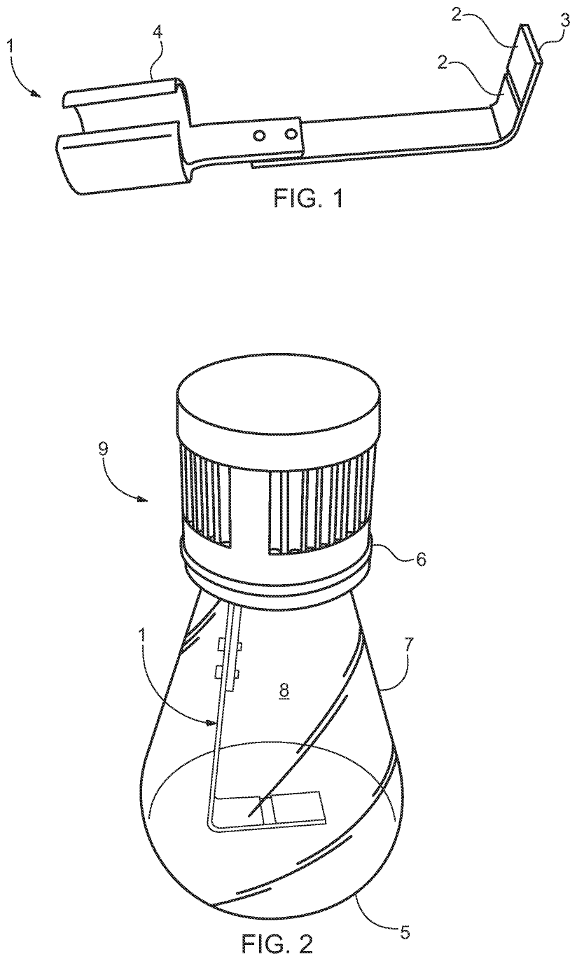

Referring to FIG. 1, in a typical cell culture incubator, a reaction vessel 112 (e.g., an incubator vessel) is used to hold cells within an incubator environment. A media is introduced into the reaction vessel to cover the cells and support growth of the cells. As the cells grow, the levels of dissolved O₂ and CO₂ change due to cell metabolism. Measuring levels of dissolved O₂ and CO₂ (or pH as a proxy for CO₂) can determine if the incubator environment has too much CO₂ and is therefore trending towards hypoxia and/or acidity, or has too much dissolved O₂ and is therefore trending toward oxygen toxicity via the cell nutrient media, for example. In accordance with an embodiment of the present invention, a sensor strip 1 can be placed within the reaction vessel so as to position the sensor within the media covering the cells. A reader 200 can be used to detect pH and/or dissolved O₂ and quantify (to a high level of accuracy) the levels at which they are present within the incubator environment. For instance, a reader can be placed outside but adjacent the reaction vessel to read the sensor 2 of the sensor strip so as to detect changes in dissolved oxygen and/or pH.

The sensor strip 1 can be a rigid or semi-rigid member (e.g., polymer, plastic, glass, metal, etc.) configured to be removably inserted into the reaction vessel 115. It is contemplated for the sensor strip to be made from clear (e.g., transparent or translucent) plastic material. The sensor strip can have at least one sensor 2 disposed or attached to a portion of the strip. The shape and configuration of the strip, as well as the placement of the sensor thereon, can be such that the sensor resides within the media when the sensor strip is inserted within a reaction vessel. For instance, the sensor strip can have a first end 3 and a second end 4, with an elongated shaft extending between the first end and the second end. The elongated shaft can be straight, bent, angled, etc. so that after the first end 3 (e.g., a distal end) is inserted into the reaction vessel, it rests on a bottom portion of the reaction vessel 5. It is contemplated for the sensor to be located at or near the first end, but it can be located at any portion of the strip. The details of the sensor and the reader will be explained later, but it is contemplated for light to be imparted on the sensor and for the sensor to emit light based on the levels of dissolved O₂ and/or pH of the media within which it is submerged. Thus, the strip portion where the sensor is located should be transparent to the anticipated (discussed in detail later) light that will be imparted to the sensor and emitted therefrom.

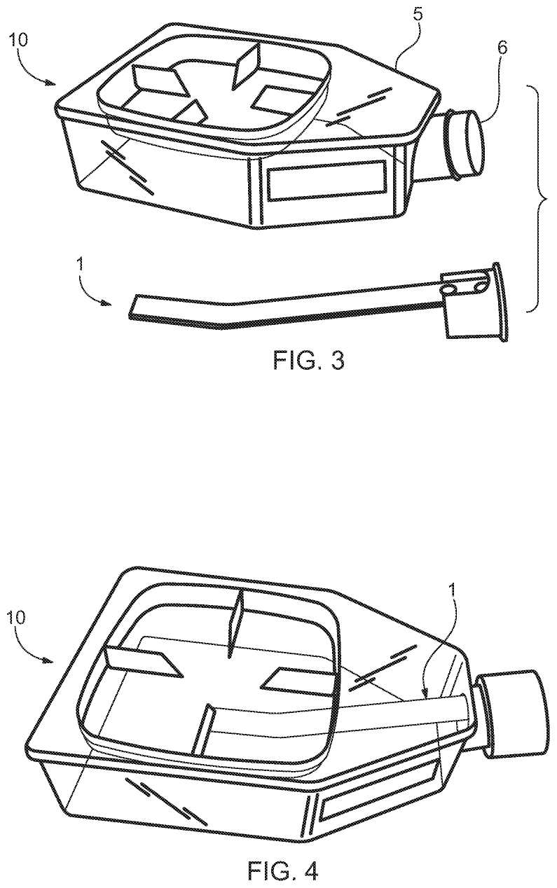

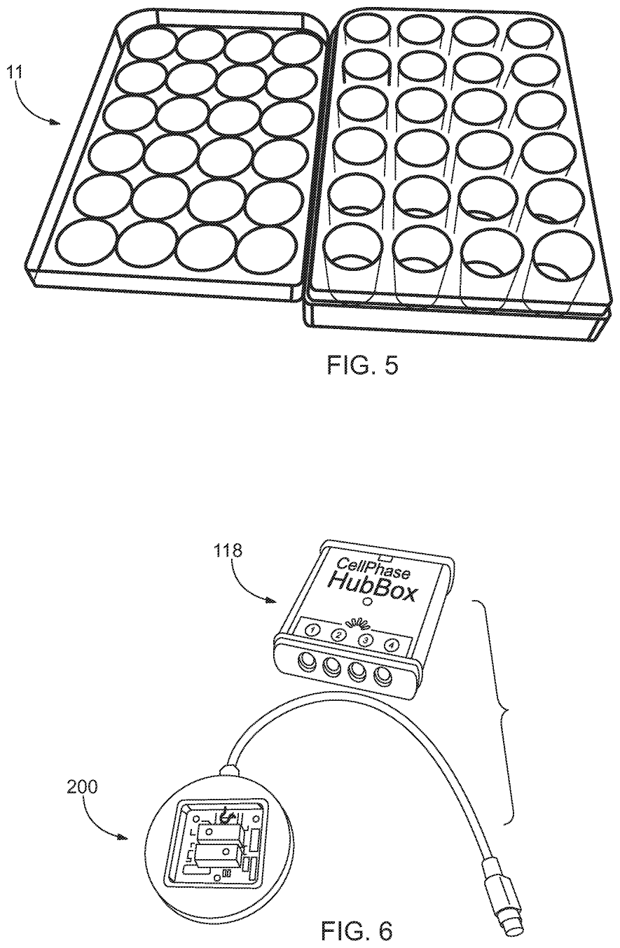

Referring to FIGS. 2-9, the reaction vessel can have an open mouth 6, a body 7, and a bottom 5 that, together, form a cavity 8. The cavity can be designed to hold the cells and the media so that the cells rest on the bottom of the reaction vessel 5 with the media residing on top of the cells. The shape and configuration of the sensor strip 1 can be designed to accommodate a standing (e.g., vertically arranged) reaction vessel 9, a sitting (horizontally arranged) reaction vessel 10, reactions vessels with or without access lids, reaction vessels with flat bottoms, contoured bottoms, bottoms with a plurality of cell culture wells 11, or any other type or style of reaction vessel. The sensor strip 1 can be configured so that the sensor 2 is located at or near the first end 3. The sensor strip 1 can be inserted through the mouth of the vessel 6 by the first end 3 spearheading the insertion. The sensor strip 1 can be inserted so that once completely inserted, the sensor 2 located at or near the first end 3 is within a volume of space defined by the media that is on top of the cells. The second end 4 (e.g., a proximal end) of the sensor strip 1 can include a stopper or plug to facilitate: prevention of the sensor strip 1 being entered too far (too far meaning that the sensor strip 1 is inserted so that the second end 4 is also in the cavity 8 and/or within the volume of space defied by the media that is on top of the cells); securing the sensor strip 1 to the mouth 6; creating a fluid (gas and/or liquid) seal between the reaction vessel mouth 6 and the sensor strip second end 4; and/or providing a handle or gripping structure by which the sensor strip 1 can be manipulated. In one embodiment, the second end 4 can be configured as a flanged-collar that can be slid into or inserted within the mouth of the reaction vessel 6 but prevents the second end of the sensor strip 4 from falling within the cavity of the reaction vessel 8.

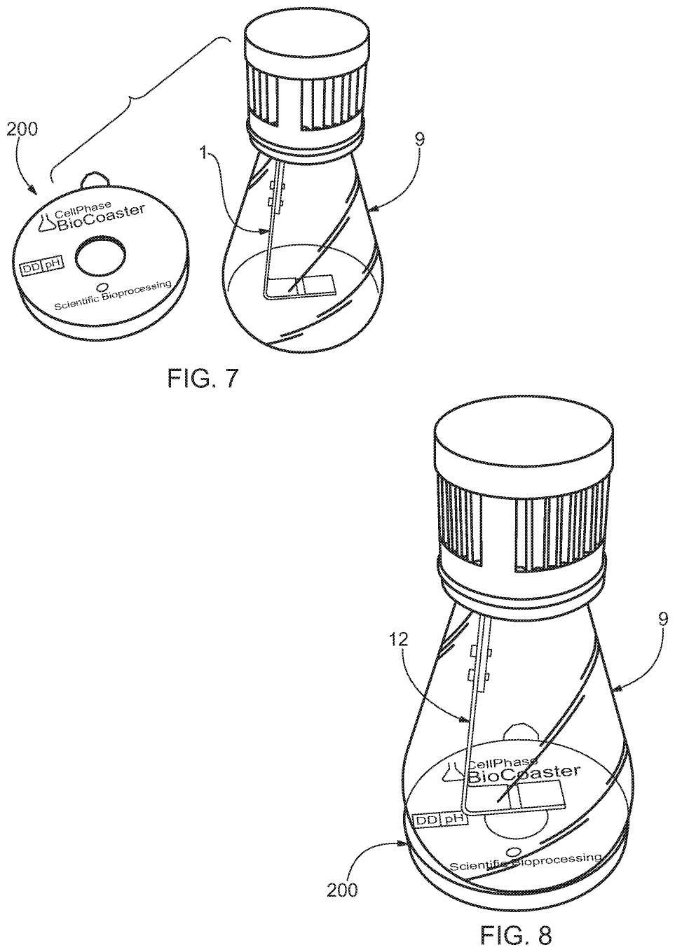

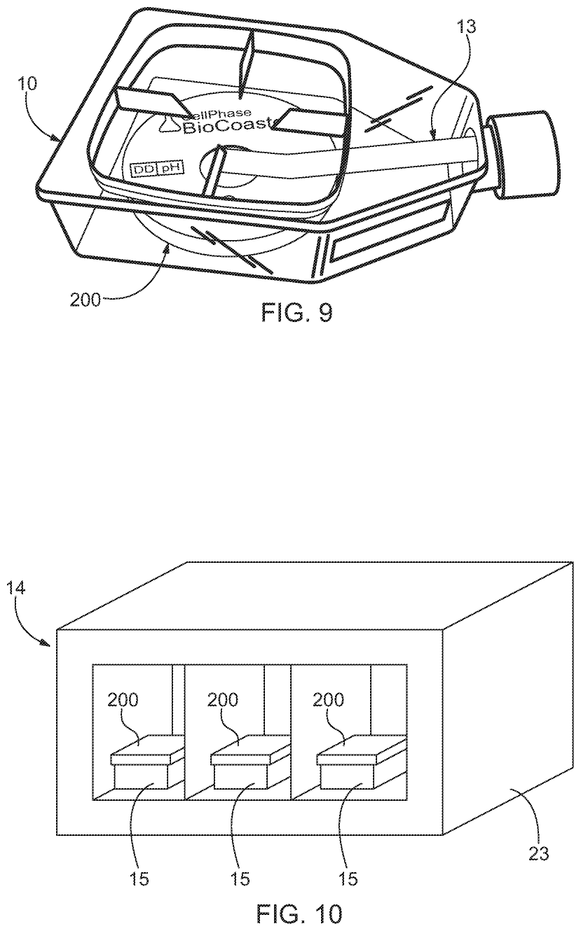

As a non-limiting example, the sensor strip can be an elongated shaft with a first end having a sensor disposed thereon and a second end having a stopper disposed thereon. The elongated shaft can extend straight from the second end and form an L-shape that leads to the first end. Such an L-shape sensor strip 12 can be used for a vertically arranged reaction vessel 9 (see FIG. 8). The L-shaped first end can be inserted through the open mouth of the vertically arranged reaction vessel until the engagement of the stopper at the second end with the vessel mouth suspends the sensor strip first end in the cavity portion of the reaction vessel so that the first end resides at the bottom of the reaction vessel or just above the bottom of the reaction vessel. The L-shape allows the first end (and the sensor) to be suspended so that it is parallel with the reaction vessel bottom, thereby allowing it to be submerged in the media. As another non-limiting example, the sensor strip can be an elongated shaft with a first end having a sensor disposed thereon and a second end having a stopper disposed thereon. The elongated shaft can extend straight from the second end and form an angled-shape that leads to the first end. Such an angled-shape sensor strip 13 can be used for a horizontally arranged reaction vessel 10 (see FIG. 9). The angled-shaped first end can be inserted through the open mouth of the horizontally arranged reaction vessel until the engagement of the stopper at the second end with the vessel mouth suspends the sensor strip first end in the cavity portion of the reaction vessel so that the first end resides at the bottom of the reaction vessel or just above the bottom of the reaction vessel. The angled-shape allows the first end (and the sensor) to be suspended so that it is parallel with the reaction vessel bottom, thereby allowing it to be submerged in the media.

In operation, the sensor strip can be sterilized and packaged into a sealed container or bag. When it is desired to have the dissolved O₂ and/or CO₂ level(s) of an incubator environment of a reaction vessel measured, a user can un-package the already sterilized sensor and insert it within the reaction vessel (obviating the need to buy expensive vessels with sensors already attached and obviating the need to sterilize the sensor before use). A reader 200 can be placed adjacent, but outside the reaction vessel. The reader 200 can be used to accurately detect the dissolved O₂ or CO₂ (or pH levels) by receiving and analyzing light being emitted from the sensor. The inventive system can provide in a quick, accurate, quantitative measurement of dissolved O₂ or CO₂ in an efficient and cost-effective manner (the details of how the sensor and the reader and can be used to generate a quantitative measurement of dissolved O₂ or CO₂ from light being emitted from the sensor will be discussed later). It should be noted that the inventive system can also obviate the need to properly align a reader with a sensor that has already been attached to the reaction vessel, as is required for existing systems.

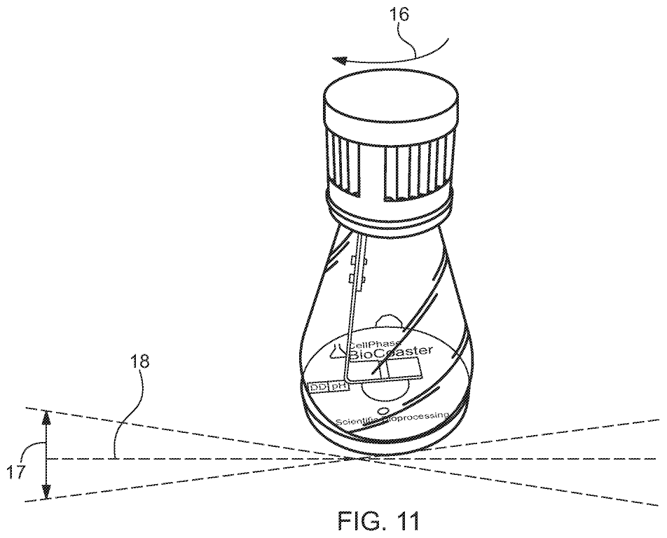

Referring to FIGS. 10-11, some embodiments can include a jig configured to secure the reaction vessel to the reader. This can be done for embodiments of the incubator systems that include a rotation unit for rotating the reaction vessel. For instance, an embodiment of the incubator system 14 can include a rotary unit 15 to cause the reaction vessel to move in a circular or precession-like manner 16 so that the media sloshes clockwise and/or counter-clockwise about the bottom of the reaction vessel. The jig can be a piece of mechanical equipment (e.g., a clamp assembly) configured to control the location and/or motion of parts associated with the rotating incubator system. Some embodiments can include a platform configured to hold a plurality of reaction vessels so as to rotate a plurality of reaction vessels (all being rotated in unison, any one or combination being rotated at different rotation rates, etc.). For instance, the system can have one rotation unit 15 to rotate the platform, or the platform can have a plurality of rotation units for a plurality of reaction vessels. The jig can be used to secure a reader 200 to an outside of a reaction vessel (or a plurality of jigs for a plurality of reaction vessels) so that the reader(s) can effectively impart and receive light to and from the sensor(s) of the sensor strip(s) that has/have been placed within the rotating reaction vessel(s).

For instance, the incubator system 14 can include a rotation unit 15 having a jig upon which the reaction vessel is placed and/or secured thereto. The incubator system 14 can be configured so that when the rotation unit 15 is activated, it causes the jig to rotate in a circular or precession-like manner 16. The jig can be a dual-clamp assembly configured to clamp to an outside portion of the reaction vessel at the bottom of the reaction vessel. The jig can also clamp a reader 200 so that it is secured to the reaction vessel at the bottom of the reaction vessel, thereby facilitating unison rotation of the jig, the reaction vessel, and the reader 200 when the rotation unit 15 is activated. When the sensor strip is inserted into the reaction vessel and the reaction vessel is caused to rotate, the reader 200 is at-all times aligned with the sensor (due to the jig(s)) so as to impart light thereon and receive light therefrom.

Some embodiments can include a rocker unit. The rocker unit can be a motor in connection with a rocker arm assembly, a cam assembly, etc. that causes the reaction vessel to rock or oscillate so as to perturb the media. The perturbation of the media can involve causing the media to slosh in a reciprocating motion in a linear direction of the rocking motion. It is contemplated to perturb the media gently so the rocking or oscillatory motion should be at a low level of angular frequency and angular displacement (e.g., rock the reaction vessel within a range 17 from −8 to +8 degrees from a horizontal position 18—the horizontal position being a 0-degree angle of displacement). Some embodiments can include a platform configured to hold a plurality of reaction vessels so as to rock a plurality of reaction vessels (all being rocked in unison, any one or combination being rocked at different angular frequencies and/or angular displacements, etc.). Again, a jig can be used to secure a reader 200 to an outside of a reaction vessel (or a plurality of jigs for a plurality of reaction vessels) so that the reader(s) 200 can effectively impart and receive light to and from the sensor(s) of the sensor strip(s) that has/have been placed within the rocking reaction vessel(s). When the sensor strip is inserted into the reaction vessel(s) and the reaction vessel(s) is/are caused to rock, the reader(s) is/are at-all times aligned with the sensor(s) (due to the jig(s)) so as to impart light thereon and receive light therefrom.

Perturbing the media gently can enhance the growth of the cells by causing the cells to be exposed to more dissolved O₂ than they otherwise would be. This can be attributed to the sloshing of the media so as to temporarily uncover the cells from the media as the reaction vessel is rocked. CO₂ is heavier or more dense than dissolved O₂ so the CO₂ generally resides as a bed of gas on the media, thereby reducing the amount of dissolved O₂ being exposed to the cells. Gently perturbing the media, however, can disturb this CO₂ bed. For instance, gently perturbing the media can cause the media (taking some or all of the CO₂ bed with it) to flow towards an end or edge of the reaction vessel, allowing for more dissolved O₂ to be exposed to the cells than otherwise would have been if the CO₂ bed would not be disturbed. This can lead to a 1.8× increase (e.g., an increase within a range from 0.1× to 1.8×) in cell growth. It should be noted that such a rocker system would benefit from using adherence cells (cells that adhere to the bottom of the reaction vessel) so that the cells remain in place at the bottom of the reaction vessel when the reaction vessel is rocked.

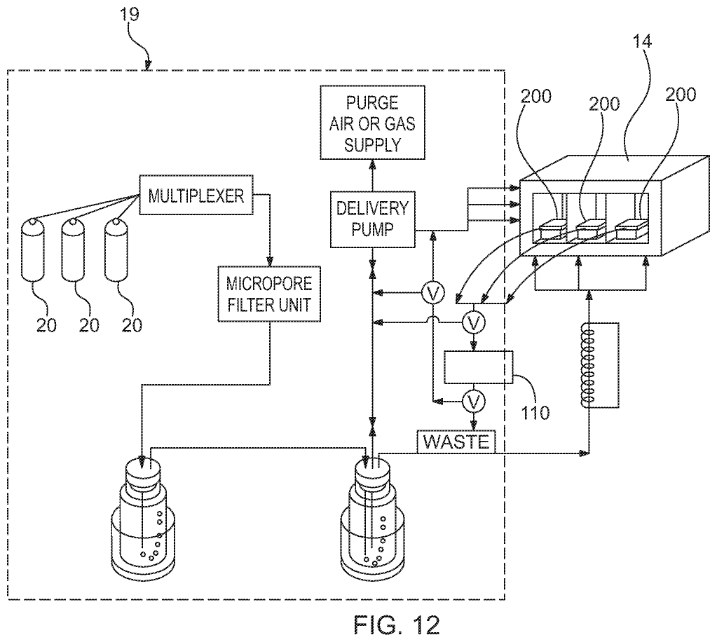

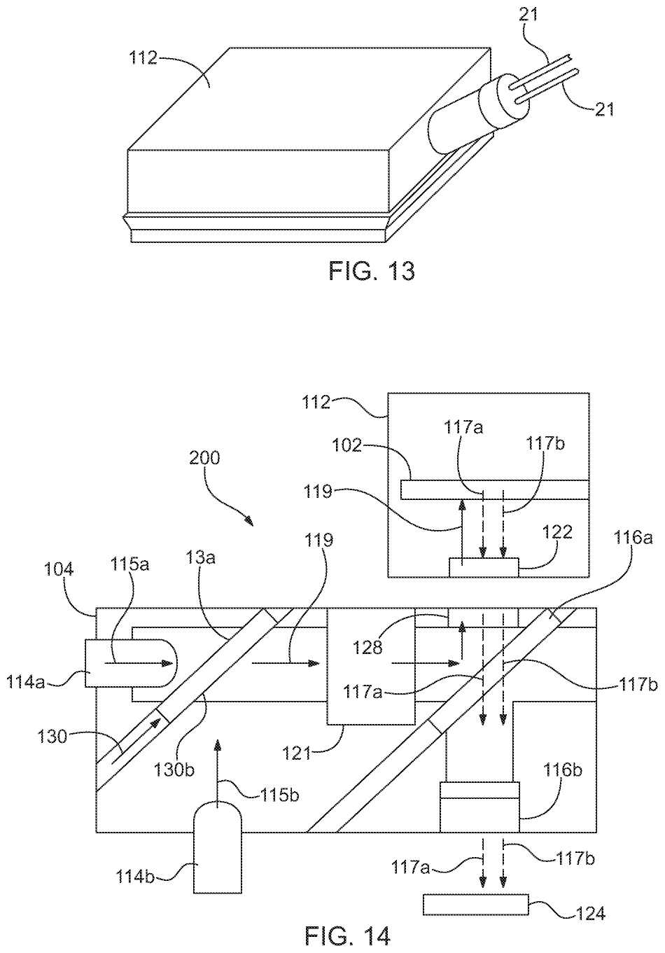

Referring to FIGS. 12-13, some embodiments of the incubator system 14 can have a gas circulation system 19 configured to adjust the CO₂ and dissolved O₂ levels in the reaction vessel 112. With conventional systems, if the CO₂ and dissolved O₂ levels were trending to undesirable levels, the media would have to be replaced. With the inventive system, the levels of CO₂ and dissolved O₂ can be adjusted instead of replacing the media. This can be achieved by the gas circulation system 19 introducing and/or removing gas from the incubation environment of the reaction vessel 112. The introduction and/or removal of gas can include gas suitable for cell growth, such as oxygen, carbon dioxide, nitrogen, or a mixture of these gases. For instance, a gas supply 20 (e.g., a gas tank) can be equipped with pumps, compressors, line or hoses, values, couplings, regulators, etc. to provide a pressurized system (e.g., positive pressure) that is a gas reservoir in fluid communication with the reaction vessel 112 and further configured to introduce gas to the reaction vessel 112. The fluid communication between the gas supply 20 and the reaction vessel can be achieved via a gas inlet/outlet 21 disposed on a portion of the reaction vessel 112. For instance, a gas inlet/outlet 21 having a gas permeable membrane can be secured to a portion of the reaction vessel 112 via a retainer ring or gasket. Gas supply lines can extend from the gas inlet/outlet 21 to the gas supply or supplies.

Some embodiments can include a platform configured to hold a plurality of reaction vessels. The gas circulation system can include a gas manifold and/or multiplexer configured to facilitate introducing and/or removing gas to and from any one or combination of the plurality of the reactions vessels. For instance, the gas supply lines from the gas inlet/outlets of the individual reaction vessels can be connected to the gas supply or supplies via the gas manifold/multiplexer. Other components such as filters, humidifiers, heaters, etc. can be used to purify and/or condition the gas before it is introduced into the reaction vessel(s). The gas in all of the reaction vessels can be adjusted in unison, the gas in any one or combination of the reaction vessels can be adjusted at different rates, concentrations, volumes, etc.).

Some embodiments can include a discharge tank equipped with pumps, compressors, line or hoses, values, couplings, regulators, etc. to provide a pressurized system (e.g., negative pressure) that is a gas reservoir in fluid communication with the reaction vessel and further configured for receiving gas from the reaction vessel. This can facilitate removal of gas from a reaction vessel. Gas supply lines can extend from the gas inlet/outlet to the discharge tank.

FIGS. 12-13 show an exemplary incubator system utilizing an exemplary reaction vessel having a gas inlet/outlet 21, and an exemplary gas circulation system 19 for supplying the reaction vessel 112 with the proper gas or gases for optimum cell growth. The system can include at least one gas supply 20. For instance, the system can include a first gas supply (e.g., a CO₂ gas tank), a second gas supply (e.g., a dissolved O₂ tank), a third gas supply (e.g., a nitrogen gas tank), etc. More of less gas supplies and other gases can be used. Each gas supply 20 can be connected to the incubator system 14 so that each gas supply 20 is connected to each reaction vessel 112 via the gas manifold and/or multiplexer 22. Supply lines or hoses from each gas supply 20 transports the gas to a manifold and/or multiplexer 22 where the gases can be directed to a particular reaction vessel 112 and/or blended together as desired for a particular application. The gas or mixed gas can then be transported to any one or combination of reaction vessels 112.

In one embodiment, the incubator system includes a housing 23 that may comprise a lid. The lid can be attached to the housing via a hinge. In some embodiments, the lid can include a seal or gasket to provide a fluid-tight seal between the lid and the housing. The housing can define a volume of space within which at least one reaction vessel is held. The housing can have the gas manifold and/or multiplexer to facilitate directional flow of the gas from the gas source(s) to the reaction vessel(s). In some embodiments, the housing can be configured to provide a positive pressure within the volume of space so as to inhibit or prevent unwanted gases from entering the housing and possibly entering the incubation environment of the reaction vessel(s). The housing can also be equipped with a heater, a humidifier, lighting, etc. to provide a conditioned environment for the cell cultures within the reaction vessel(s) placed therein.

It should be noted that embodiments of the system can be configured to have any one or combination of the platform to accommodate a plurality of reaction vessels, the rotation unit, the rocker unit, or the gas circulation system. In addition, any embodiment of the system can include a sensor strip and a reader for detecting and measuring levels of CO₂ and dissolved O₂. As will be explained herein, the incubator system can include a computer device. This can be done to generate a computer system. With the computer device and/or computer system, a feedback loop can be created between the reader, the gas circulation system, the incubator housing, the rotary unit, and/or the rocker unit to monitor the CO₂ and dissolved O₂ levels in real-time and to adjust the CO₂ and dissolved O₂ levels, rocking, rotation, temperature, humidity, lighting, pressure, etc. in response thereto. This can include monitoring the CO₂ and dissolved O₂ levels of each reaction vessel individually or in unison, as well as adjusting adjust the CO₂ and dissolved O₂ levels, rocking, rotation, temperature, humidity, lighting, pressure, etc. of each reaction vessel individually or in unison.

Some embodiments can be configured as a kit. For instance, a kit can include one or more sensor strip(s) with one or more of reaction vessel(s) and/or one or more reader(s).

A detailed discussion of embodiments of the sensor and the reader will be discussed net.

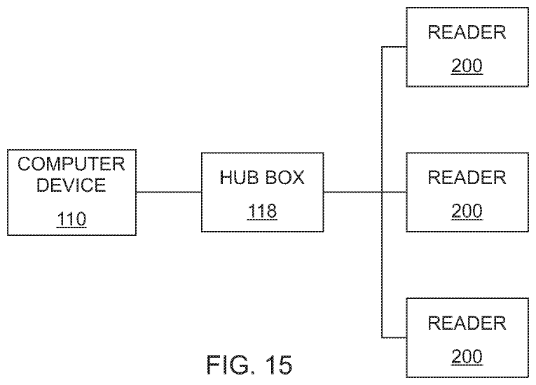

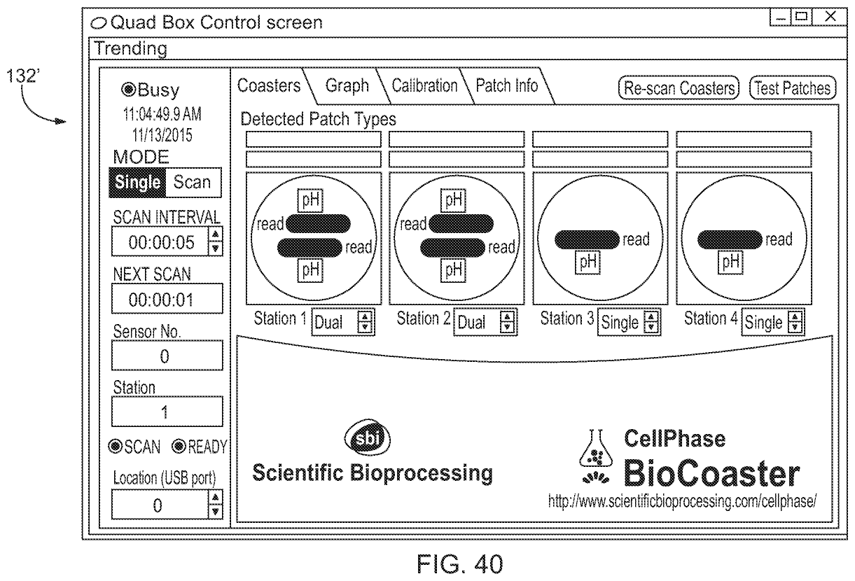

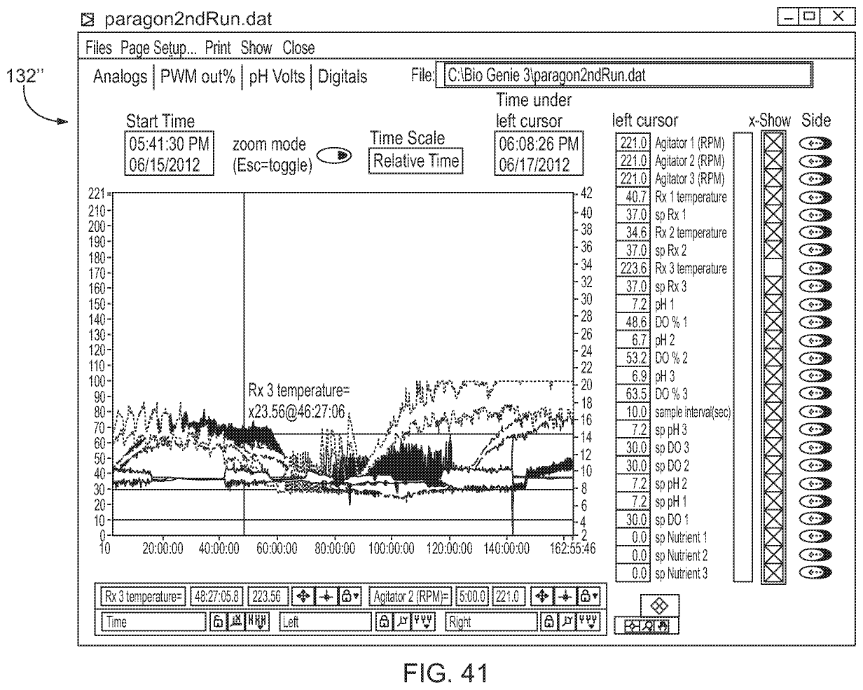

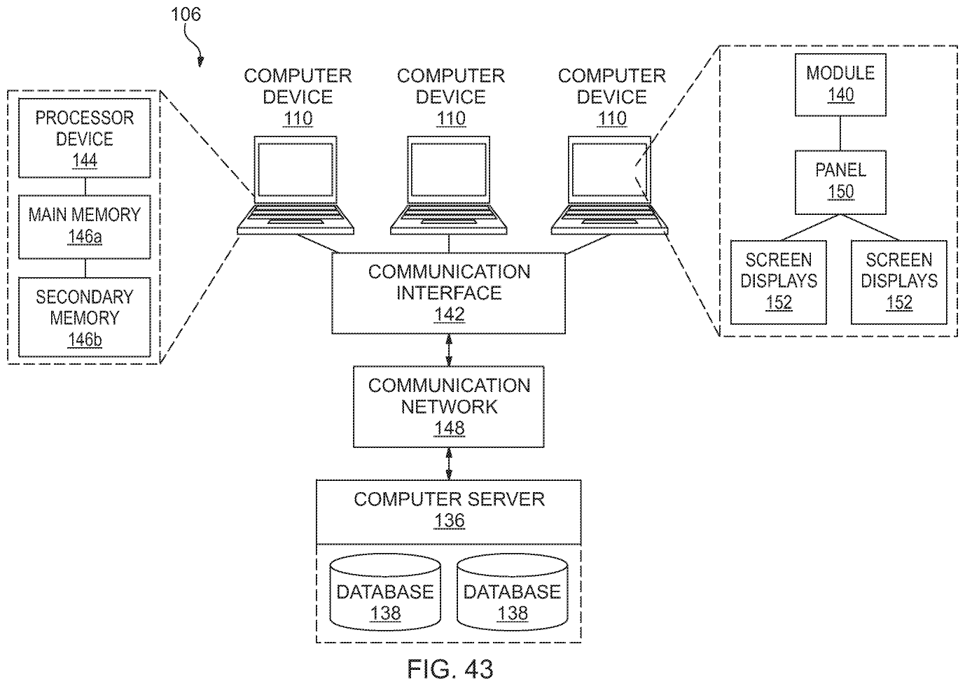

Referring to FIGS. 14-15, the reader can include a beam combiner assembly 104, for example. Some embodiments can include at least one computer device 110 in connection with the reader 200 (see FIG. 15). The computer device 110 can be programmed to run software generating user interfaces 132′, 132″ (see FIGS. 40-41) that may be displayed via the computer device 110. The computer device 110 may be part of a computer system 106 (see FIG. 43). Some embodiments include use of a sensor patch 102 (e.g., a fluorescent-based patch) as the sensor. The beam combiner assembly 104 can include at least one illumination source 114 a, 114 b optical filters/mirrors 116 a, 116 b, and/or other optoelectronics, which can be used to generate excitation light beams 115 a, 115 b and detect emitted light 117 a, 117 b that may be induced by fluorescence. A hub box 118 (e.g., a data acquisition card) can be used to communicatively and operably associate the computer device to the beam combiner assembly. Use of a hub box 118 can facilitate data transmission between the computer device 110 and one or more beam combiner assemblies 104.

In some embodiments, at least one hub box 118 can be used to acquire data from a plurality of reaction vessels 112. In further embodiments, the hub box 118 can be used to determine a life of the patch 102, which may be defined as the time frame by which the patch 102 can effectively fluorescence. This can be done by acquisitioning code data from the patch 102. For instance, the code can be manufacturing data, for example, that may be transmitted to the computer device 110 to calculate expiration times. Other data, such as an expected offset of a patch 102 can be encoded within the code that is associated with the patch 102. This code can include information such as a date of manufacture of the patch 102, a date at which the expected offset was determined, a date the patch 102 was packaged, etc. Chemicals within the patch 102 can photo-bleach after a statistically pre-determined use has elapsed (e.g., imparting excitation light beams on the patch 102 every fifteen seconds for ninety days can cause chemicals impregnated into the patch 102 to fail to effectively fluoresce and radiate emitted light), and thus the code can include an expected expiration date based on the date of manufacture. In another embodiment, the computer device 110 can calculate the expected expiration date based on such data.

The code can further include a “calibration” date, which can include a pre-set time period after which a date the expected offset was determined. In some embodiments, the “calibration” date can indicate when the expected offset should be determined again. For example, if the date the expected offset is determined by a computer device 110 to be greater than six months from a current date of use, then the computer device 110 may transmit a signal to the hub box 118 indicating that the patch 102 should be “calibrated.” The hub box 118 can further be structured to have at least one useful life and/or calibration light indicator, indicating gradations of approaching expiration and/or calibration dates for the patch 102. For example, a green indicator light can be used to indicate that the patch 102 has at least two months before replacement and/or calibration. An amber light can be used to indicate that the patch 102 has less than one month before replacement and/or calibration. A red light can be used to indicate that the patch 102 has less than one week before replacement and/or calibration. Other indicator light schemes and/or time frames can be used.

In some embodiments, the computer device 110 can be communicatively and operably associated with a computer system 106 via a computer network. (See FIG. 43). In one embodiment, the hub box 118 can enable collection of data associated with each reaction vessel 112 being monitored and enable command data to be transmitted to each beam combiner assembly 104 from a user of the computer device 112 via the user interface 108 (see FIGS. 40-41) of the system 100. In some embodiment, each reaction vessel 112 and/or computer device 110 can be in connection with the hub box 118 via a hardwire and/or via wireless connection. The hardwire connection can be can be achieved via a USB cable, other data cable, coaxial cable, T1 cable, or other network cable. The wireless connection can be achieved via transmitter, receiver, and/or transceiver units, which may be in connection through a communications network 148. Embodiments including the computer system 106 can facilitate data transfer to and from a plurality of computer devices 110, hub boxes 118, and/or reaction vessels 112. Controllers and actuators can be used to influence pumps, photodiodes, etc. that may be used with embodiments of the system via command data transmitted through the computer device(s) 110. In some embodiments, the computer device 110 can be programmed to generate command data automatically based on algorithms, which can be based on reaction vessel data being collected, as well as command data entered by a user via a user interface 132′, 132″ of the computer device 110 in communication with the system.

At least a portion of a reaction vessel 112 can be transparent to form a reaction vessel window 122. In some embodiments, light 115 a, 115 b generated by a beam combiner assembly 104 can be directed to be incident upon a patch 102 located within a reaction vessel 112. The reaction vessel window 122 can be further configured to allow emitted light 117 a, 117 b from induced fluorescence of the patch 102 to be passed through the reaction vessel 112 so that it can be incident upon a detector 124 of the reader 200 that may be located outside of the reaction vessel 112, which may include being located on an outside surface of the reaction vessel 112 or on an outside surface of the beam combiner assembly 104. The detector 124 can be a device that is configured to detect various emitted light 117 a, 117 b based on wavelength and/or intensity. In some embodiments, the detector 124 can be a photodiode. In further embodiments, the detector 124 can also include a processor configured to convert the detected light into emitted light data, which can be representative of the light being detected. The detector 124 may also be configured to transmit the emitted light data to another component of the system.

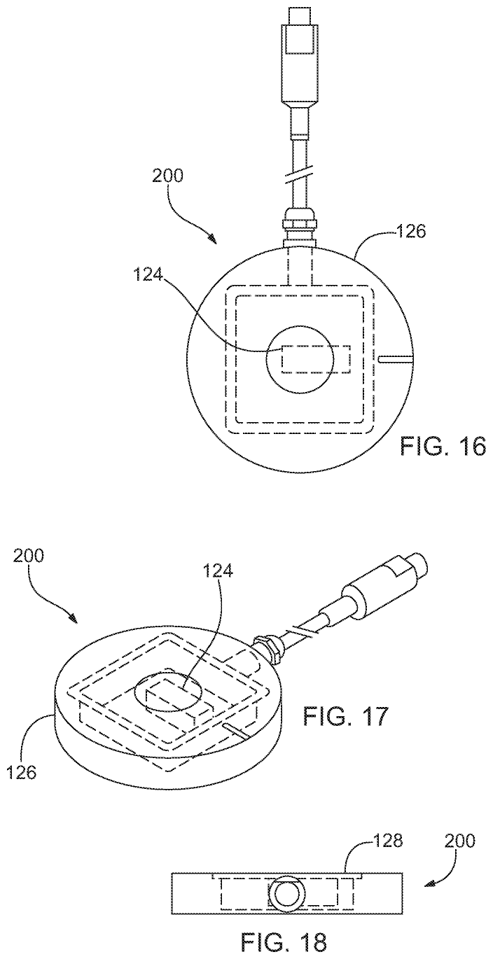

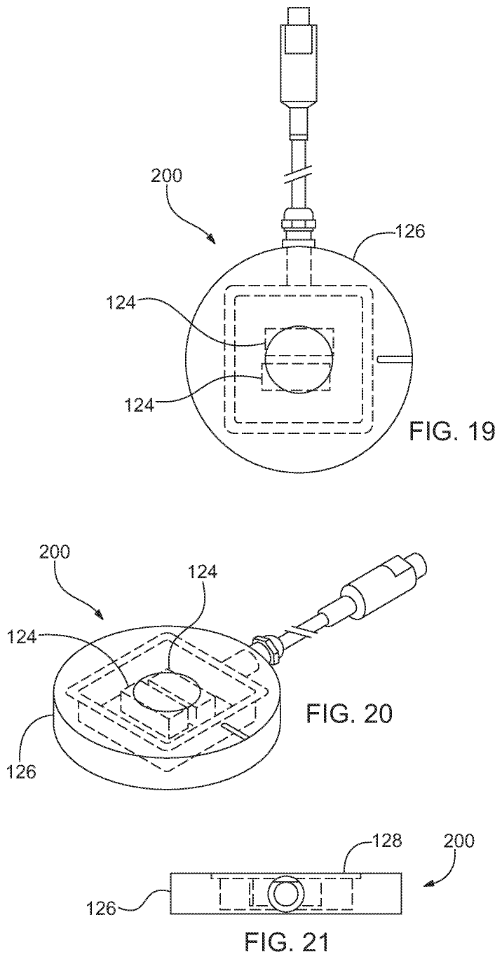

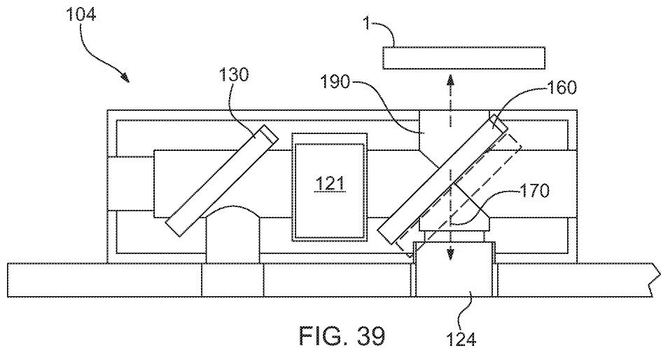

Referring to FIGS. 16-21, the beam combiner assembly 104 can include the detector 124. In some embodiments, both the beam combiner assembly 104 and the detector 124 can be housed within a casing 126. The casing 126 can be configured to allow the reaction vessel 112 to be placed thereon or adjacent thereto. In some embodiments, the casing 126 can be coaster shaped. For example, the casing 126 can be a planar puck-like object, which may have a profile that is round, square, triangular, etc. In one embodiment, the casing 126 can include a rigid puck-like body with a beam combiner assembly window 128 through which excitation and/or emitted light can travel. The beam combiner assembly window 128 may include an aperture 190 (see FIG. 39) within a body of a casing 126. The aperture 190 may further include a transparent cover and/or lens to enable transmission of excitation 115 a, 115 b and/or emitted light 117 a, 117 b, but prevent foreign objects from entering into a casing 126. For example, the transparent cover may be a filter configured to permit certain wavelengths of light and/or bands of wavelengths to be transmitted there-through. In at least one embodiment, the beam combiner assembly window 128 can be structured with a widened aperture so as to facilitate more excitation and/or emitted light to pass there-through (see FIG. 39). Some embodiments can include a beam combiner assembly 104 with a single detector 124 or single sensing unit (see FIGS. 16-18) and some embodiments can include a plurality of detectors 124 or a plurality of sensing units (see FIG. 19-21).

The beam combiner assembly 104 may house illumination sources, filters, mirrors, detectors, and other electro-optics. Generation of excitation light beams 115 a, 115 b may be achieved through use of at least one light emitting diode (“LED”), laser, or other illumination source 114 a, 114 b capable of generating coherent light and/or light within a very narrow bandwidth of wavelengths. Direction of excitation light beams 115 a, 115 b and/or emitted light 117 a, 117 b within a beam combiner assembly 104 can be achieved through use of waveguides, reflectors, refractors, etc.

In at least one implementation, the beam combiner assembly 104 can be positioned outside a reaction vessel 112 with a reaction vessel 112 placed adjacent thereto (e.g., placed on top) so that excitation light beams 115 a, 115 b generated by a beam combiner assembly 104 can be incident on the patch 102 within a reaction vessel 112 when caused to transmit through the reaction vessel window 122. For example, the beam combiner assembly 104 can facilitate resting a beam combiner assembly 104 on a flat, stable surface, which may enable placing a reaction vessel 112 on top and adjacent the beam combiner assembly window 128. For example, the reaction vessel 112 can be placed on top of the beam combiner assembly 104, where at least one excitation light beam 115 a, 115 b can be directed through a bottom of the reaction vessel 112 to be incident upon the patch 102 that may be attached to an inside surface of a reaction vessel 112.

The system may include a plurality of beam combiner assemblies 104. Whether there is one beam combiner assembly 104 or more than one, each beam combiner assembly 104 can be placed into electrical connection with an electrical power source to enable operation of the illumination sources 114 and/or other electro-optics. Any beam combiner assembly 104 can be further placed into electrical connection with the hub box 118. The hub box 118 can be separately placed into electrical connection with an electrical power source. As excitation light beams 115 a, 115 b are generated and directed to the patch 102 to induce emitted light 117 a, 117 b due to fluorescence, the emitted light 117 a, 117 b can be detected by the detector(s) 124. Excitation light beams 117 a, 117 b can be generated in a controlled manner via the computer device 110 (e.g., via algorithms and/or command data from users of a computer device). Emitted light data can be generated by the detector 124 and transmitted via the hub box 118 to the computer device 110 for data processing, data manipulation, and/or data analysis. In some embodiments, the detector 124 can include a processor to facilitate digitization of the emitted light 117 a, 117 b and transmission of digitized signals to the hub box 118 and/or the computer device 110.

In the non-limiting exemplary embodiment shown in FIGS. 14-15, an exemplary beam combiner assembly 104 can include at least one illumination source 114 a, 114 b, at least one filter/mirror arrangement 116 a, 116 b, at least one beam combiner 130, and at least one detector 124. The first illumination source 114 a can generate a first excitation light beam 115 a (e.g., a violet light beam at or near 405 nm). The second illumination source 114 b can generate a second excitation light beam 115 b (e.g., blue light beam at or near 470 nm). Each of the first and second excitation light beams 115 a, 115 b can be directed toward the beam combiner 130. The beam combiner 130 can be positioned (e.g., at a desired angle incident to each of the first and second excitation light beam 115 a, 115 b) and structured (e.g., dichroic filter/mirror) to pass one of the excitation light beams 115 a, 115 b and reflect another excitation light beam 115 a, 115 b at an angle so that both excitation light beams 115 a, 115 b are coaxial and collimated in a single direction. For example, a violet light beam 115 a can be directed toward a beam combiner 130 at a given angle (e.g., 45-degree angle), incident upon a first surface 130 a of the beam combiner 130, whereas a blue light beam 115 b can be directed toward the beam combiner 130 at a given angle (e.g., 45-degree angle), incident upon a second surface 130 b of the beam combiner 130. The first surface 130 a of a beam combiner 130 can be a light filter (e.g., a dichroic filter), and the second surface 130 b of the beam combiner 130 can be a reflector (e.g., a dichroic mirror). The violet light beam 115 a can be allowed to pass through while the blue light beam 115 b may be reflected and caused to travel in a same direction as the violet light beam 115 a, thereby generating a combined violet-blue light beam 119 that is collimated and coaxial. Other filters and/or mirror configurations, as well as angles of incidence and reflection can be used for the beam combiner 130. In some embodiments, excitation light beams 115 a, 115 b can be made to refract or even diffract from components of the beam combiner 130 to generate a combined light beam 119 comprising the first and second excitation light beams 115 a, 115 b that is collimated and coaxial.

The combine light beam 119 can be directed toward the first filter/mirror arrangement 116 a, which may be a dichroic filter/mirror. The combined light beam can be further sharpened before being incident upon the first filter/mirror arrangement 116 a by a beam sharpener 121. The first filter/mirror arrangement 116 a can reflect the combined light beam 119 and cause it to travel through the beam combiner assembly window 128. The combined light beam 119 can then be directed through the reaction vessel window 122 to be incident upon the patch 102 that may be located within the reaction vessel 112. The combined light 119 being incident upon the patch 102 can induce fluorescence of chemicals within the patch 102. This may cause at least one emitted light 117 a, 117 b to be generated and radiate from the patch 102. The emitted light 117 a, 117 b be can at a certain wavelength, which may depend on the pH level and/or DO level of the environment within which the patch 102 is exposed. For example, with a combined light beam 119 comprising violet and blue light, chemicals impregnated into the patch 102 can be configured such that they emit green light 117 a from the patch 102 so as to be indicative of changes in pH of a growth medium. In some embodiments, emitted light 117 a of a certain wavelength (e.g., green light) can be generated when the pH level is below or above a threshold level. As another example, chemicals impregnated into the patch 102 can be configured such that emitted light 117 b of a certain wavelength (e.g., red light) from the patch 102 can be generated so as to be indicative of changes in DO of a growth medium. In some embodiments, emitted red light 117 b can be generated when the DO level is below or above a threshold level. Thus, a first emitted light 117 a of a certain wavelength can be generated that is indicative of pH level, and a second emitted light 117 b of a certain wavelength can be generated that is indicative of DO level.

An example of a DO sensor patch 102 can include a use of ruthenium-based oxygen sensing films such as Ru(II) tris (4,7-diphenyl-1,10-phenanthroline) complex, immobilized in a silicone rubber membrane (Bambot, S. B. et al., Biotechnol. Bioeng. 43: 1139-1145 (1994)). Another example of a DO sensor patch 102 can include impregnating a material with an indicator dye such as a porphyrin dye, for example, or a metalloporphyrin such as platinum(II)-octaethyl-porphyrin combined with, e.g., encapsulated within, a polymer matrix such as polystyrene. The matrix layer may then be applied to a polystyrene support using, for example a toluene-based solvent (Liebsch, G. I. et al., Appl. Spectroscopy 54: 548-559 (2000)).

An example of a pH sensor patch 102 can include impregnating a material with any known ratiometric pH sensitive dye, such as 1-hydroxypyrene-3,5,7-sulfonic acid (HPTS). A sterilized solution of the dye can be directly introduced into a bioreactor media and detected via fluorescence. Fluorescence detection can be determined using front face geometry. For example, HPTS has two excitation peaks-400 and 450 nm. When excited at either 400 or 450 nm, HPTS can emit light at approximately 520 nm. The longer excitation peak can be excited using a blue LED (460 nm), for example, and the shorter excitation peak can be excited using an UV LED (375 nm), for example. The intensity ratio of the 520 nm fluorescence emissions from excitation at each of the two excitation peaks can be affected by the pH of the media. Thus, the pH can be calibrated by measuring the intensity ratio of the 520 nm fluorescence emissions at each of the two excitation peaks as the pH changes. pH can be optionally verified on a benchtop pH meter. This ratiometric approach may avoid interference from turbidity changes and provides accurate measurements of pH.

Other examples of pH and DO sensor patches 102 can be based on techniques disclosed in U.S. Pat. No. 6,673,532, filed Aug. 14, 2001, titled “Bioreactor and Bioprocessing Technique,” which is incorporated herein by reference in its entirety.

Emitted light 117 a, 117 b from the patch 102 can travel back through the reaction vessel window 122 and further through the beam combiner assembly window 128. The emitted light 117 a, 117 b can be further directed to be incident upon the first filter/mirror arrangement 116 a. It is contemplated for the emitted light 117 a, 117 b (e.g., light emitted due to fluorescence) to be generally at a wavelength that is higher than a wavelength of any of the excitation light beams 115 a, 115 b required to elicit the fluorescence effect. Thus, emitted light traveling 117 a, 117 b back toward the first filter/mirror arrangement 116 a may have wavelengths that are greater than both of the first and second excitation light beams 115 a, 115 b. In at least one embodiment, a surface of first filter/mirror arrangement 116 a can be configured to reflect the combined excitation light beams 119, but to pass emitted light beams 117 a, 117 b. For example, the surface of the first filter/mirror arrangement 116 a can be configured to pass emitted red and/or green light coming in-through the beam combiner assembly window 128, but reflect combined violet-blue light beams so as to be directed out-through the beam combiner assembly window 128.

The emitted light 117 a, 117 b can be further directed to be incident upon a second filter/mirror arrangement 116 b. As shown in FIGS. 14-15, a pass filter can be generated by both the first filter/mirror arrangement 116 a and the second filter/mirror arrangement 116 b. In some embodiments the pass filter can be a long pass filter. The long pass filter can be configured for passing light with certain wavelengths, or light with wavelengths greater than a minimum wavelength. For example, with the violet and blue excitation light beams 115 a, 115 b and the green and red emitted light beams 117 a, 117 b, the long pass filter may be configured to pass light with wavelengths greater than 525 nm. The long pass filter can be used by the beam combiner assembly 104 to block any light below the minimum wavelength defined by the pass filter. For example, the long pass filter can be used by the beam combiner assembly 104 to block light that is at and/or below a wavelength of the emitted light 115 a, 115 b. This may be done to prevent any stray excitation light 115 a, 115 b from passing through the long pass filter, which may be used to prevent any excitation light 115 a, 115 b from being detected by the detector 124. For instance, the long pass filter can be used by the beam combiner assembly 104 to block any light having a wavelength that is at and/or below 525 nm (e.g., block the violet and/or blue excitation light), but allow passage of any emitted light 117 a, 117 b (e.g., allow the green and/or red emitted light). Emitted light 117 a, 117 b can then be directed to be incident upon the detector 124. Data signals from the detector 124 may then be used to record wavelengths and/or intensities of the emitted light 117 a, 117 b. The recorded wavelengths and/or intensities can be associated with changes in pH and/or DO levels. For example, wavelengths at and/or near the first emitted light 117 a (e.g., the green light) can be indicative of a pH level at a threshold level and/or a decreasing pH level. Wavelengths at and/or near the second emitted light 117 b (e.g., the red visible light) can be indicative of DO levels at a threshold level and/or increasing DO levels. Intensities of emitted light 117 a, 117 b can be indicative of an amount of pH and/or DO. Recording intensities as a function of time can be used to determine or calculate rates of change of pH level and/or DO level.

While various embodiments disclose use of two excitation light beams 115 a, 115 b and two emitted light beams 117 a, 117 b, these are one exemplary. There can be any number of excitation 115 and emitted light beams 117 used. Further, the excitation 115 and emitted light beams 117 are not limited to the specific wavelengths disclosed, but the specific wavelengths are only exemplary of what can be used.

The sensor patch 102 can be a fluorescent-based optical patch. In one embodiment, the patch 102 can include a substrate with a polymeric backing. The substrate can be filter paper, which may be a monomer (e.g., methyl methacrylate) or cellulose-based filter paper. In some embodiments, the backing can include a silicon-based backing. The backings and/or the substrate can be configured to facilitate quick absorption of the excitation light beams 115 a, 115 b and quick radiation of emitted light beams 117 a, 117 b. One way to achieve this is by combining at least two fluorescent dyes in a monomer while depositing a measured amount of the same onto an inner surface of the reaction vessel 112. The measured amount can also be polymerized. It should be noted that the water solubility of polymers may vary, and thus polymer selection for the backing may be difficult. It should be further noted that the activity of chemical constituents of polymers can vary widely. Because of this potential variability, pre-calibration of the fluorescent response of a polymetric spot can be difficult to perform. However, using filter paper as a matrix for the reactive chemicals and dyes can facilitate easier calibration. Using filter paper as a matrix for the reactive chemicals and dyes can further increase the rate at which a response may be generated.

In one exemplary embodiment, a sterile optical patch 102 can be placed aseptically in the reaction vessel 112. The back of the patch 102 can be coated with an adhesive for adhering it to an inside surface of the reaction vessel 112. The adhesive can be a biocompatible adhesive. For example, silicone based adhesives with no support binders have been shown to be biocompatible. The patch 102 can be placed within the reaction vessel 112 so as to enable at least one excitation light beam 115 a, 115 b generated from the beam combiner assembly 104 to be incident upon it through a transparent portion of a reaction vessel 112. For example, the patch 102 can be placed adjacent the reaction vessel optical window 122 and/or at a position covering the reaction vessel optical window 122. In one embodiment, the patch 102 can be placed at a position subtending the reaction vessel optical window 122.

The transparent portion of the reaction vessel 112 can be the optical window 122 of the reaction vessel 112 configured to allow at least certain wavelengths of light (e.g., wavelengths associated with excitation light beams 115 a, 115 b and/or emitted light beams 117 a, 117 b) to transmit there-through. This may include blocking all other light from passing there-through, or blocking certain bands of light (wavelength bands). The patch 102 can include chemicals configured to generate at least one emitted light beam 117 when caused to fluoresce due to at least one excitation light beam 115 b eing incident upon it. This can be achieved by, for example, impregnating the patch 102 with a blend of chemicals to generate emitted light 117 when subjected to at least one excitation light beam 115 and/or a combined light beam 119 of at least two excitation beams 115. Chemicals within the patch 102 can be further configured to radiate at least one the emitted light 117 in response to changes in oxygen partial pressure and/or pH levels. In some embodiments, the patch 102 can be at least one of a pH patch and a DO patch. A pH patch can be configured to radiate emitted light 117 at a certain wavelength in response to changes in pH levels. A DO patch can be configured to radiate emitted light 117 at a certain wavelength in response to changes in DO levels.

The pH patch can be structured to generate a ratio-metric response. For example, the chemicals impregnated into the pH patch may be excited by two different, but close, excitation light beams 117 (e.g., different with respect to wavelengths). This can cause generation of two different emission light beams 117 (e.g., each having a different wavelength). Each wavelength of the different emitted light beams 117 can differ depending on changes in pH levels the pH patch is exposed to. The ratio between wavelengths of the different emission light beams 117 can be used as an indicator of the pH level of a growth media in the reaction vessel 112.

The DO patch can be a fluorescent oxygen-sensing patch, which can be structured to use oxygen as a quenching agent to quench a chemical response of chemicals impregnated into the DO patch while exposed to a presence of oxygen. The DO patch can be further structured to radiate emitted light when excited by a single excitation light beam 117. The DO patch can be further structured to generate an emitted light beam 117 as a function of the chemical response. In some embodiments, the DO patch can be structured such that the more oxygen that is present in the environment within which the DO patch is located, the less the chemical response occurs. This may lead to a weaker emission light beam signal. Thus, the less oxygen that is present, the greater the chemical response occurs. This may lead to a stronger emission light beam signal generated by the system. Thus, the lower the levels of DO within a culture sample, the stronger the signal that can be detected from the emitted light. Generally, oxygen content of the growth media in a culture growth bioprocess is less than that of ambient air. Therefore, enabling generation of strong signals in environments where oxygen content is within a range from greater than 0% to 21% can be beneficial for cell culture monitoring.

The system can be configured so that a wavelength of an excitation light 115 a beam to elicit a fluorescence response from a DO patch can be lower than a wavelength of an excitation light beam 115 b to elicit a fluorescence response from a pH patch. For example, a wavelength of an excitation light beam 115 a to elicit a response from a pH patch can be blue light beam (e.g., at or near 470 nm) and a wavelength of an excitation light beam 115 b to elicit a response from a DO patch can be violet light beam (e.g., at or near 405 nm).

A mini-fluorometer can be used to generate excitation light beams 115 a, 115 b and to detect wavelengths of emitted light 117 a, 117 b. For example, the mini-fluorometer can be built into an integrated circuit board 134, which can be placed into communication with the computer device 110 via a hub box 118. Optoelectronics, such as a photodiode (e.g., the detector 124) for example, can be used to interrogate the patch 102 via modulation of excitation light beams 115 a, 115 b to detect the emitted light 117 a, 117 b. In some embodiments, a mini-fluorometer can be built into an integrated circuit board 134, both of which can be attached to and/or placed within the casing 126 forming the beam combiner assembly 104.

In at least one embodiment, an illumination source 114 for generating at least one excitation light beam 115 can be an LED. A band filter can be used to produce a narrow bandwidth of excitation light 115 coming from the illumination source 114. The band filter can be further used so that the gain of an LED may be adjusted to generate a desired intensity.

The sensor patch 102 can be mediated by changes in pH levels and/or changes in DO levels in growth media that support growth of cells within the reaction vessel 112. Detected emitted light 117 radiating from the patch 102 can be captured as signals and digitized by the detector 124. The digitized signals can be transmitted as reaction vessel data to the computer device 110. The computer device 110 can be located within and/or outside of a bio-safety laboratory. With reaction vessel data, the computer device 110 can be programmed to calculate fluorescence lifetimes and decay rates associated with oxygen concentration. This can be done to calculate DO concentration within a growth medium of cell culture sample. A first user interface 132′ can be displayed on the computer device 110, which may be programmed to cause the computer device 110 to display instantaneous values of DO concentration (See FIG. 40). A second user interface 132″ can be programmed to cause the computer device 110 to display a time course of the calculated DO concentration and/or pH levels. For example, FIG. 41 shows time source data of pH units between 5.50 and 8.50.

The computer device 110 can be programmed to influence electrical, mechanical, and optical components of a system. This may include, but is not limited to, influencing controlling valves, pumps, mixers, detection devices, etc. For example, software can be stored on the memory 146 a, 146 b of the computer device 110, which may be programmed to cause the computer device 110 to accept reaction vessel data and/or to accept command data from users via the user interface 132′, 132″. Command data can include threshold levels and operating parameters. The software can be further programmed cause the computer device 110 to drive components of the system automatically within user-defined thresholds and user-defined parameters. In addition, a user can set pre-established rules to influence threshold levels and operating parameters to generate variations within each reaction vessel 112. In some embodiments, the system can be configured so that each reaction vessel 112 only responds to one set of parameters and thresholds, each of which can be pre-determined by a user. Thus, a group of parameters and thresholds can be set for a plurality of reaction vessels 112. For example, a group of parameters and thresholds can be set for as many as twelve reaction vessels 112. Any of the reaction vessels 112 can be arranged in parallel with another reaction vessel 112.

Some embodiments can include use of firmware as an alternative or in addition to software. In at least one embodiment, a digital sensing board (“DSB”) in connection with the system can include firmware programmed such that at least one computation is performed on the DSB. This can be done to have the DSB perform certain calculations and/or functions (e.g., addition of a gas to the reaction vessel 112) as opposed to the computer device 110 running software performing that calculation and/or function. In at least one embodiment, the DSB can be incorporated into a bioprocessor so that a signal can be transmitted from the DSB to a pump, a valve, or other system component. The signal can be transmitted directly from the DSB to the system component. Thus, actions performed by the system component can be initiated by the DSB and/or the computer device 110. In some embodiments, certain actions by the system component can be initiated by the DSB without being initiated by the software. This may be done to eliminate use of a computer device 110 for some or all aspects of the system. In some embodiment, monitoring functions and/or initiated action by certain monitoring functions can be customized by use of the software.

In at least one embodiment, the combined excitation light beam 119 can be collimated and coaxial. Further, the patch 102 can be placed adjacent the beam combiner assembly 104 so that it is subtending the detector 124. The beam combiner assembly 104 may be structured to combine at least two different excitation light beams 115. The beam combiner assembly 104 can be further structured to compare two emitted light 117 waves for pH analysis, and use intensity of a third emitted light 117 wave for DO analysis. The system can be further configured to detect emitted light 117 with reference to an expected offset. This may allow use of the system without a user knowing and/or without a user having to pre-setting the system to accommodate: 1) which excitation light beams 115 are being generated and/or which emitted light beams 117 are being induced; 2) whether the patch 102 is a pH patch or a DO patch; and/or, 3) whether the reaction vessel 112 with both a pH patch and a DO patch is being used. Hence, a reaction vessel 112 containing a pH patch and/or a reaction vessel 112 containing a DO patch can be used at any time without having to calibrate and/or re-calibrate the system.

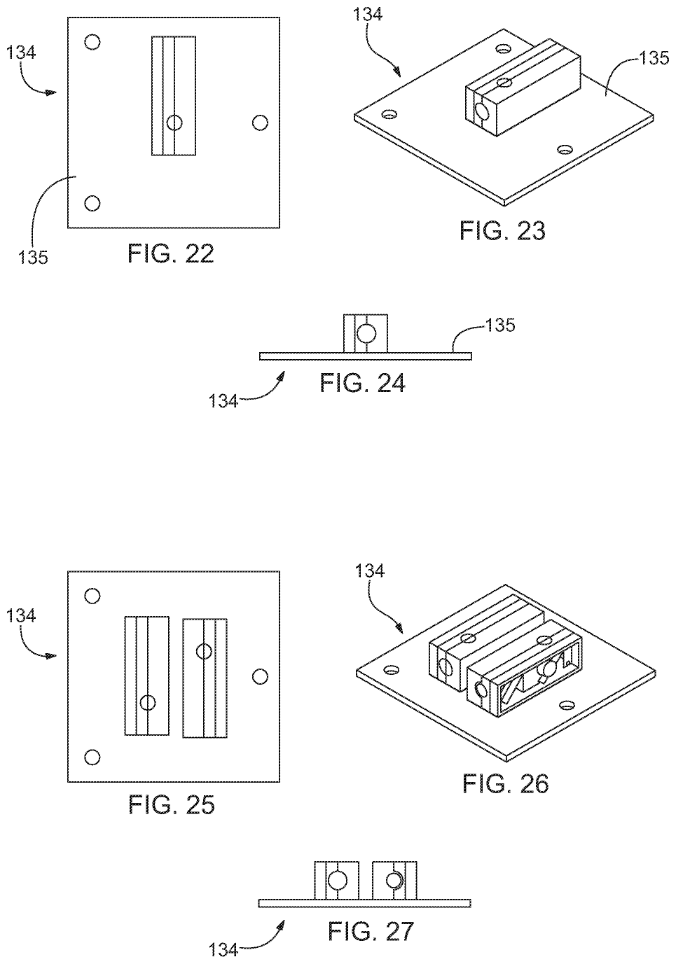

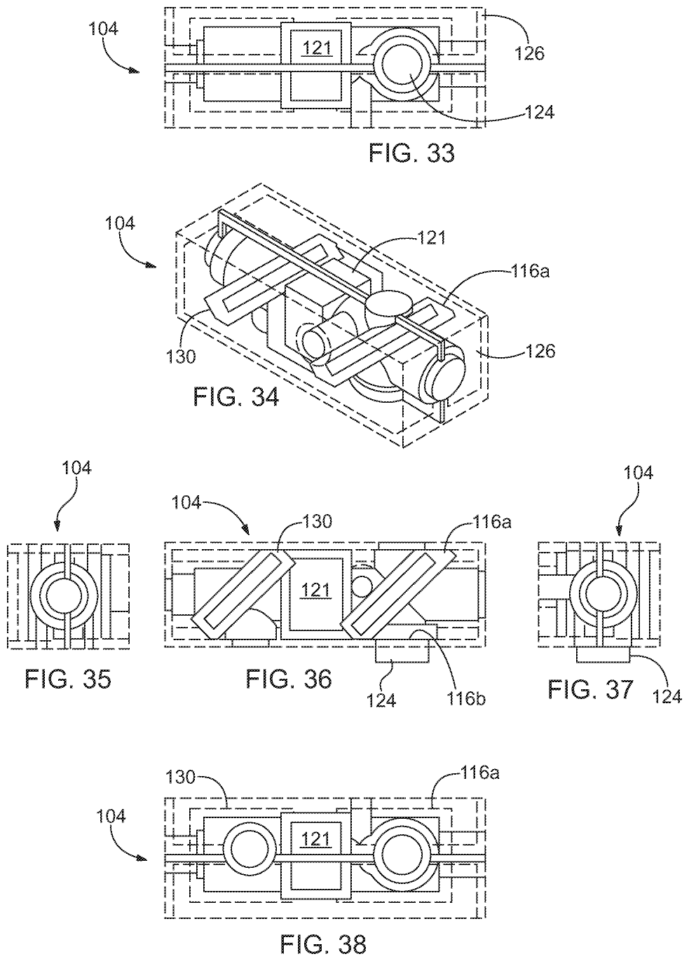

Referring to FIGS. 22-39, various views of exemplary beam combiner assemblies 104 are disclosed. In at least one embodiment, the beam combiner assembly 104 can include an array of four dichroic filter/mirrors within a circuit board block 134 as part of the pass filter. Some embodiments can include circuit board blocks 134 with a single detector 124 or single sensing unit (see FIGS. 22-24) and some embodiments can include circuit board blocks 134′ with a plurality of detectors 124 or a plurality of sensing units (see FIG. 25-27).

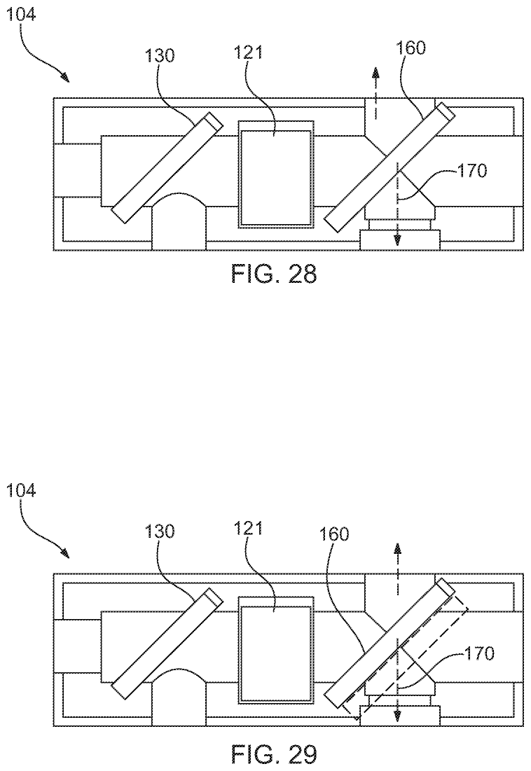

Setting a filter/mirror within a base 135 of a circuit board 134 and shifting a mirror of a filter/mirror arrangement 116 can increase available space within the circuit board block 134, which can enable improvements within the system. Referring to FIGS. 28-29, shifting the mirror 160 of at least one filter/mirror assembly 116 can increase the available space within the circuit board block 134. Shifting the mirror 160 can also cause coincidence with an optical axis 170. For instance, FIG. 29 shows the mirror 160 that is configured to reflect the combined light beam 119 through the beam combiner assembly window 128 where the mirror 160 is shifted so that the reflected combined light 119 coincides with the optical axis 170 of beam combiner assembly window 128. In contrast, FIG. 28 shows an un-shifted mirror 160 that reflects the combined light beam 119 so that only a portion of the combined light 119 is transmitted through the beam combiner assembly window 128.

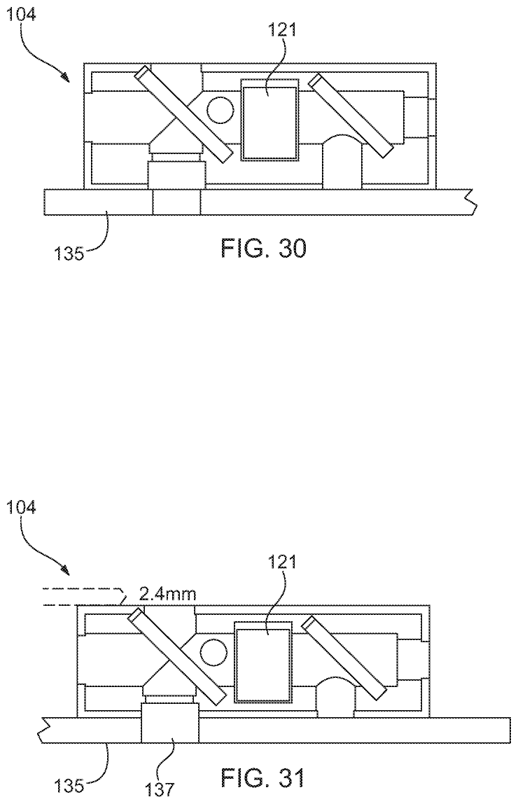

FIGS. 30-31 show the reduction in space of the circuit board block 134 that may also be achieved with use of a cylindrical shaped filter/mirror 137. Setting the filter/mirror within the base 135 of the circuit board 134 can be achieved with use of a cylindrical shaped filter/mirror 137. Cylindrical shaped filters/mirrors 137 may not only reduce space within the circuit board block 134, but they can also be easier and less costly to set within the circuit board block 134 from a manufacturing stand-point than other shaped filters/mirrors. Thus, the cylindrical shape of the filter 137 can make it easier to set it into the circuit board 134, especially during mass production. FIG. 30 shows a circuit board block 134 with a non-cylindrical shaped filter/mirror. FIG. 31 shows an embodiment of the circuit board block 134 with the cylindrical shaped filter/mirror 137. With the exemplary embodiment of FIGS. 30-31, a reduction of 2.4 mm can be achieved via use of the cylindrical shaped filter/mirror 137. The reduction may be achieved by allowing the cylindrical filter/mirror 137 to extend at least partially into the base 135 of the circuit board block 134.

In at least one embodiment, the system can include a filter/mirror arrangement 116 with a shifted mirror, where shifting the mirror can facilitate use of the cylindrical filter 137. In some embodiments, the shifted mirror 160 configuration can further allow the cylindrical filter 137 to protrude into the base 135 of the circuit board block 134 to which it may be attached. In some embodiments, the cylindrical filter 137 may extend below the base 135. This may enable reducing a height of the circuit board 134 even further, which may create additional room at the top of the circuit board 134. Reducing space can be beneficial, because the system and/or the reaction vessel 112 may be placed within an incubator. The space within an incubator may be compromising, and thus a reduction in volume occupied by the system can be beneficial.

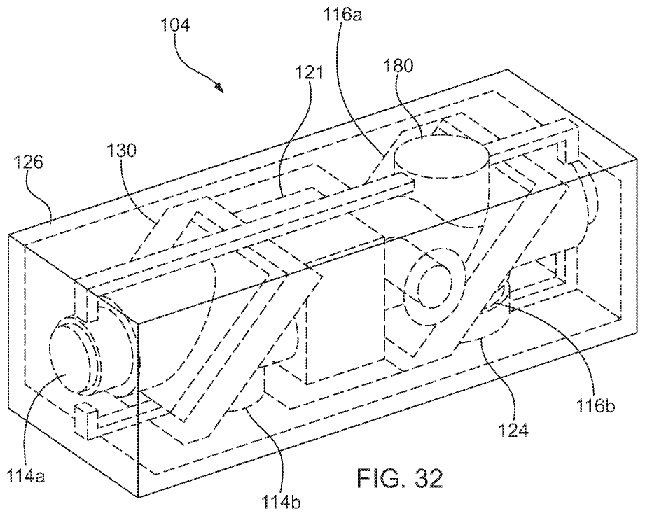

Referring to FIGS. 32-38, in some embodiments, increasing available space within the circuit board block 134 can facilitate placement of an offset-setting LED inside the circuit board block 134. As described earlier, an expected offset can be determined with use of a red reflector and/or a red LED. Any one of these may be referred to as an offsetting generator 180. In other words, the red reflector and/or the LED can be used to facilitate determining an expected offset by generating a light beam with a wavelength corresponding to the expected offset. The LED can be used as an alternative to the reflector or in addition to the reflector. Creating available space can facilitate placing the LED and/or reflector inside of the casing 126. Note that the LED and/or reflector can be any color LED or reflector that may correspond to the wavelength of light associated with the expected offset. Thus, the LED or reflector need not be a red.

Determining an expected offset can be achieved as follows. An empty reaction vessel 112 can be examined via the system. The red reflector can be placed against the beam combiner assembly window 128 so that red light can be detected by the system. Alternatively, a red LED can be included within the circuit board block 134 of the beam combiner assembly 104. The beam combiner assembly 104 can generate excitation light beams 115 so as to detect red light by the detector 124, the red light being generated by the offsetting generator 180. This detected red light can be used as an expected offset. As noted above, the offset generator need not be red, but the detected red light can be used as an expected offset with embodiments where the presence of DO is expected to radiate red light by a DO patch. It should be noted that the chemical response for a DO patch can be linear. Thus, the red light of the red LED or red reflector can simulate the expected red emitted light of a DO patch when in use within the reaction vessel 112 and when DO is present within the reaction vessel 112.

Using an expected offset can enable use of any reaction vessel 112 without knowing beforehand whether the reaction vessel 112 has a pH patch or a DO patch. This can further enable use of the system without performing calibration of filter/mirror positions and/or angles. For example, the system can be encoded with the expected offset before using any patch 102 to conduct monitoring (e.g., the expected offset can be encoded to be used by software operated by the computer device 110). During use with the patch 102, the system can detect emitted light 117, and if the emitted light 117 is at or near the expected red light (i.e., the expected offset) then the detection system 100 can automatically determine that a DO patch is being used. If during use with the patch 102, the system detects emitted light 117 that is not at or near the expected offset, then the system can automatically determine that a pH patch is being used. The expected offset can be determined for each patch 102, or a set of patches 102, and encoded to be associated with each patch 102, or set of patches 102. Each patch 102, or set of patches 102, can be associated with the expected offset before being used in a system for monitoring cell cultures.

In at least one embodiment, the system can be used in the following manner. An empty reaction vessel 112 without the patch 102 can be placed in line with an excitation path of the system such that there is no reflected (fluoresced) light reaching the detector 124. The red reflector 180 can be placed against a beam combiner assembly window 128. Alternatively, a red LED 180 can be included within the circuit board block 134 of the beam combiner assembly 104. The reaction vessel 112 can then be placed adjacent the beam combiner assembly 104 so as to be between the beam combiner assembly 104 and the red reflector 180 and/or red LED 180. The beam combiner assembly 104 can be caused to generate excitation light beams 115 so as to detect red light by the detector 124, the red light being generated by the reflector offsetting generator 180. Alternatively, the LED offsetting generator 180 can be used to generate the red light. The detected red light can be used to determine an expected offset. The expected offset can then be record. The recorded expected offset can be associated with a plurality of patches 102, the plurality including at least one of a pH patch and a DO patch, so that an expected offset can be known for users of a set of patches 102. The expected offset associated with each patch 102 and/or set of patches 102 can be recorded and coded. A coded expected offset associated with each patch 102 and/or set of patches 102 can be placed within a barcode associated (e.g., attached thereto) with the reaction vessel 112 and/or patch 112, stored as a scatter code, stored within software of the computer device 102, transmitted to the computer device 102 from another computer device 102 via the computer system 106, etc.

A pH patch and/or a DO patch can be affixed to an inside of the reaction vessel 112 with the reaction vessel 112 being empty otherwise. A cell culture sample can be placed within the reaction vessel 112 for which an expected offset for the patch 102 associated therewith has been calculated. The red reflector offsetting generator 180 can be removed from a beam combiner assembly 104 and/or the red LED offsetting generator 180 can be turned off. At least one of a violet excitation light beam 115 a (e.g., at or near 405 nm) and a blue excitation light beam 115 b (e.g., at or near 470 nm) can be generated to be combined into a combined violet-blue excitation light beam 119 by the beam combiner 130. The combined violet-blue excitation light beam 119 can include the combined violet excitation light beam 115 a and a blue excitation light beam 115 b, which may be combined to be collimated and coaxial. The combined violet-blue excitation light beam 119 can be directed through the beam combiner assembly window 128 and further through the reaction vessel window 122 to be incident upon at least one of a pH patch and a DO patch. The combined violet-blue excitation light beam 119 can induce fluorescence from chemicals within at least one of the pH patch and the DO patch so that emitted light 117 a, 117 b is radiated therefrom and directed back through the reaction vessel window 122 and further through the beam combiner assembly window 128 to be incident upon the pass filter. The pass filter can pass light with wavelengths at or greater than 525 nm so as to be incident upon the detector 124, but block light with wavelengths less than 525 nm.

The detector 124 can digitize the emitted light 117 a, 117 b being incident upon it. The detector 124 can also record at least one of wavelength and intensity of the emitted light. The detector 124 can also be configured for generating a representative data detection signal therefrom. The detection signal can be transmitted to the hub box 118. The hub box 118 can be in communication with at least one beam combiner assembly 104, which can be in further communication with at least one computer device 110 (see FIG. 15). The coded expected offset for the reaction vessel 112 and/or patch 102 can be entered into the computer device 110 for the reaction vessel 112 and/or a patch 102 or a set of reaction vessels 112 and/or set of patches 102. The computer device 110 can then process, manipulate, and store detection signals for monitoring conditions of the cell growth medium of a cell culture within each reaction vessel 112 transmitting data to the system.

If during detection of emitted light 117 a, 117 b, an expected offset is detected, the computer device 110 can be programmed to determine that a DO patch is being used, a threshold level of DO is present, and/or a DO level is increasing or decreasing. If during detection of emitted light 117 a, 117 b, no expected offset is detected, the computer device 110 can be programmed to determine that a pH patch is being used, a threshold level of pH is present, and/or a pH level is increasing or decreasing. The reaction vessel 112 can be removed from the beam combiner assembly 104 and a different reaction vessel 112 can be placed on the beam combiner assembly 104 for DO level and pH level monitoring without calibration of filter/mirror positions and/or angles.

The sensor strip 1 from a same set can be switched multiple of times without calibration of filter/mirror position and/or angle and without entering a coded expected offset. If a reaction vessel 112 from a different set is used, monitoring can be done without calibration of filter/mirror positioning and/or angle, but a coded expected offset associated with those set of patches 102 and/or reaction vessels 112 may have to be entered.

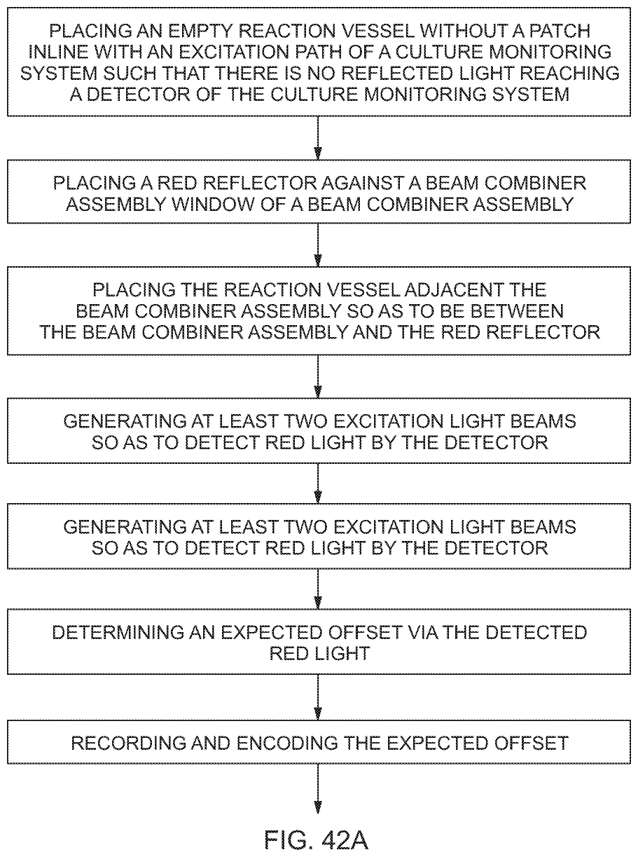

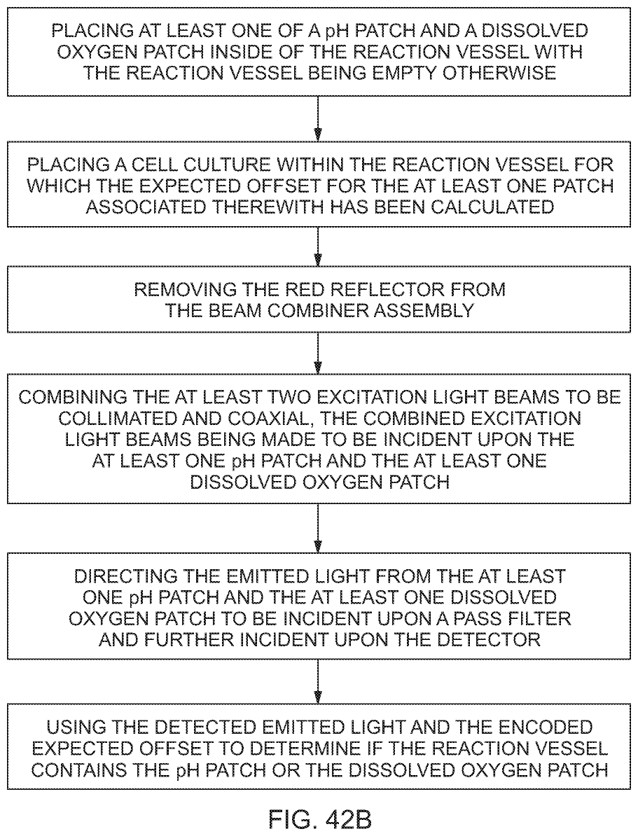

Referring to FIGS. 42A-42B, a method of using the detection system 100 can include monitoring cell cultures. The method can include placing an empty reaction vessel 112 without a patch 102 in line with an excitation path of a culture monitoring system such that there is no reflected light reaching the detector 124 of the culture monitoring system. The method can further include placing a red reflector offsetting generator 180 against a beam combiner assembly window 128 of a beam combiner assembly 104. The method can further include placing the reaction vessel 112 adjacent the beam combiner assembly 104 so as to be between the beam combiner assembly 104 and the red reflector offsetting generator 180. The method can further include generating at least two excitation light beams 115 a, 115 b so as to detect red light by the detector 124, the red light being generated by the red reflector offsetting generator 180. The method can further include determining an expected offset via the detected red light. The method can further include recording and encoding the expected offset. The method can further include affixing at least one of a pH patch 102 and a dissolved oxygen patch 102 to an inside of the reaction vessel 112 with the reaction vessel 112 being empty otherwise. The method can further include placing a cell culture within the reaction vessel 112 for which the expected offset for the at least one patch associated therewith has been calculated. The method can further include removing the red reflector offsetting generator 180 from the beam combiner assembly 104. The method can further include combining the at least two excitation light beams 115 a, 115 b to be collimated and coaxial, the combined excitation light beams 119 being made to be incident upon the at least one pH patch 102 and the at least one dissolved oxygen patch 102. The method can further include directing the emitted light 117 a, 117 b from the at least one pH patch 102 and the at least one dissolved oxygen patch 102 to be incident upon a pass filter and further incident upon the detector 124. The method can further include using the detected emitted light and the encoded expected offset to determine if the reaction vessel 112 contains the pH patch 102 or the dissolved oxygen patch 102.

In further embodiments, a feedback loop can be generated via the system and at least one computer device 110 so that reaction vessel data can be transmitted to the computer device 110 and command data can be to be transmitted to at least one beam combiner assembly 104 from a user of the computer device 110 via the user interfaces 132′, 132″ of the system (see FIGS. 40-41 and 43). Furthermore, controllers and actuators can be used to influence pumps, photodiodes, etc. of the system via command data transmitted through the computer device 110 and/or the DSB. The computer device 110 and/or DSB can be programmed to generate command data automatically based on algorithms, which can be based on reaction vessel data being collected. Additionally, or in the alternative, command data can be entered by a user via the user interface 132′, 132″ of the computer device 110 in communication with the system.

Other configurations and uses for the reader 200 can be any of the embodiments disclosed in U.S. Pat. No. 10,379,047, the entire contents of which is incorporated herein by reference.

### Exemplary Computer System Architecture

As shown in FIGS. 40-41 and 43, the system can include a computer system 106 programmed to generate at least one user interface (“UI”) 132′, 132″ displayed on a display unit of at least one computer device 110. Data entered via a module of the UI 134 can be transmitted to the computer system 106 for processing and storage Data acquisitioned from a system, a database of the computer system 106, and/or any other computer device 110 of the computer system 106 may be transmitted to the computer system 106 to be manipulated by a processor device for generating functional aspects of various user interfaces 132′, 132′ that can be displayed by any of the computer devices 110 in communication with the detection system 100. Wherever a user is referenced in this disclosure, it is understood that this reference includes computer device(s) 110, computer server(s) 136, and/or database(s) 138 associated with the user's use thereof. Distributed communication networks used to enable connection and communication between each computer device 110 may include communications in whole, or in part, via web-sites through at least one communication network, which may include a web-server.

The computer system 106 may include a plurality of computer devices 110, computer servers 136, databases 138, communication networks 148, and/or communication path/connections. A user of the system may use at least one processor device 144, memory storage 146 a, 146 b, and communications interface 142 to communicate and execute commands. Each computer server 136 may be connected to at least one database 138, where software executed by each computer device 110 may carry out functions of storing, coalescing, configuring, and/or transmitting data. Software may be stored on any type of suitable computer-readable medium or media. This may be a non-transitory computer-readable medium or media, such as a magnetic storage medium, optical storage medium, or the like.

The computer system 106 architecture shown in FIG. 43 is an exemplary embodiment of a computer system 106 that may be used to facilitate interactions between computer devices 110, at least one DSB, users of computer devices 110, and the computer system 106, which may be implemented using hardware, software, firmware, non-transitory computer readable media having instructions stored thereon, or any combination thereof, and may be implemented in a single or multiple of computer systems 106 or other processing systems. Hardware, software, or any combination thereof may embody software and/or hardware modules 140 and/or components used to execute functions of the computer system 106 and/or a system. If programmable logic is used, such logic may execute on a processing platform or a special purpose device. Embodiments of the disclosed subject matter can be practiced by using various computer system configurations, including multi-core multiprocessor systems, minicomputers, mainframe computers, computers linked or clustered with distributed functions, and/or pervasive or miniature computers that may be embedded into virtually any device. For instance, the computer device 110 can include a processor device 144 operably associated with a memory 146 a, 146 b. Any computer device 110 may be used to implement any disclosed embodiment of the invention.

The processor device 144 may be a single processor, a plurality of processors, or combinations thereof. The processor device 144 may have one or more processor cores. The processor device 144 may be a special purpose or a general purpose processor device. The processor device 144 may be connected to a communication infrastructure. The communication infrastructure may include, but is not limited to, a bus, message queue, network, multi-core message-passing scheme, etc.

The computer device 110 may include a main memory 146 a. The main memory 146 a may include, but is not limited to, a random access memory, a read-only memory, etc. The computer device 110 may include a secondary memory 146 b. The secondary memory 147 b may include, but is not limited to, a hard disk drive, a removable storage drive, a floppy disk drive, a magnetic tape drive, an optical disk drive, a flash memory, etc. Any of the main and the secondary memories 146 a, 146 b can be a non-volatile memory.

Computer program media, non-transitory computer readable media, and computer usable media may refer to tangible media, such as, for example, a removable storage unit and a hard disk installed in a hard disk drive. The removable storage drive may read from and/or write to a removable storage unit. The removable storage unit can include a removable storage media that can be read by, and written to, a removable storage drive. For example, if a removable storage drive is a floppy disk drive, the removable storage unit may be a floppy disk. The removable storage unit can also be non-transitory computer readable recording media.

In some embodiments, the secondary memory 146 b may include alternative means for allowing computer programs or other instructions to be loaded into the computer device 110 and/or computer system 106. This may be, for example, a removable storage unit and/or an interface. Examples of such means may include, but are not limited to, a program cartridge and cartridge interface (e.g., as found in video game systems), a removable memory chip (e.g., Electronic Erasable Readable Programmable Read-Only Memory (“EEPROM”), Programmable Read-Only Memory (“PROM”)), etc. and associated socket, and/or other removable storage units and interfaces.

The computer system 106 may include a communications interface 142. The communications interface 142 may be configured to allow software and data to be transferred between computer devices 110 within the computer system 106 and/or the computer system 106 and external devices. Communications interfaces 142 may include, but are not limited to, a modem, a network interface (e.g., an Ethernet card), a communications port, a Personal Computer Memory Card International Association (“PCMCIA”) slot and card, etc. Software and data transferred via a communications interface may be in a form of signals, which may be electronic, electromagnetic, optical, or other signals. Signals may travel via a communications path, which may be configured to carry signals and may be implemented using wire, cable, fiber optics, a phone line, a cellular phone link, a radio frequency link, etc.

Computer program medium and computer usable medium may refer to memories, such as a main memory and a secondary memory, which may be memory semiconductors (e.g., Dynamic Random-Access Memory (“DRAM”)). These computer program products may be means for providing software to the network. Computer programs (e.g., computer control logic) may be stored in a main memory 146 a and/or a secondary memory 146 b. Computer programs may also be received via the communications interface 142. Such computer programs, when executed by a processor device 144, may enable a computer device 110 to execute commands and act upon the various components of the system 106. Accordingly, such computer programs may represent controllers of the computer system 106, where software may be stored in a computer program product and loaded into the computer device 110 using a removable storage drive, an interface, a hard disk drive, and/or a communications interface 142.

In some embodiments, the computer device 110 include a processor, a microprocessor, minicomputer, server, mainframe, laptop, personal data assistant, a cellular phone, smartphone, pager, or any other programmable device configured to enable transmission and/or reception of data, which may be over a network. The computer device 110 may include a peripheral device, such as an input/output device. The peripheral device may include, but is not limited to, a keyboard, a mouse, a screen display, a touch screen, a stylus pen, a monitor, a printer, a hard disk drive, a floppy disk drive, a joystick, an image scanner, etc.

One or more electronic communication networks 148 may be utilized by the computer system 106 to promote communication among different components, transfer data, and/or share resource information. Such communication networks 148 may be embodied as, but not limited to, at least one of Ethernet, wireless Local Area Network (“LAN”), Mobile Area Network (“MAN”), Wide Area Network (“WAN”), Virtual Private Network (“VPN”), Storage Area Network (“SAN”), Global Accelerator Network (“GAN”), Home Phoneline Network Alliance (“HomePNA”), etc.

In some embodiments, the computer system 106 may include a computer device 110 configured as a processor 144 operatively associated with at least one module 140, which may be programmed to display panels 150 and/or screen displays 152 on a monitor of a computer device 110. The processor 144 may be programmed to execute computer-readable instructions included within the module 140. Computer-readable instructions may be in a form of software stored on a non-transitory computer readable medium operatively associated with the processor 144. Each module 140 may be configured to generate the user interface (“UI”) 132′, 132″, which may enabling at least one user to issue commands, access data stored on a data storage media operatively associated with the processor, and/or transmit data to and from the data storage media. The module 140 may include software, firmware, hardware, or any reasonable combination thereof.

Any of the panels 150 may be programmed to display information and grant access to data related to certain aspects and functionalities of the computer system 106 and/or a system. Through the various modules 140 and panels 150, the computer system 106 can provide a communication network to orchestrate interaction between a user, the computer system 106, and the various components of the system. For instance, different panels 150 of each module 140 may be programmed to facilitate differentiated display of information and differentiated interaction between users, components of a computer system, and components of the system. This may be achieved by each module 140 generating different UIs 132′, 132″ for control of different aspects of the system.

Various embodiments of the present disclosure can be described in terms of the example computer system 106 described herein; however, other embodiments of the computer system 106, along with other embodiments of computer architectures, can be used. Although operations may be described as a sequential process, some of the operations may be performed in parallel, concurrently, and/or in a distributed environment, and with program code stored locally or remotely for access by single or multi-processor machines.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

What is claimed is:

1. A sensor strip, comprising:
a strip comprising a first end and a second end;
the first end having a fluorescence-based sensor disposed or attached thereto, the first end being further configured to be inserted into a reaction vessel; and
the second end having a stopper configured to engage with a mouth of the reaction vessel so as to secure the sensor strip to the reaction vessel and to position the fluorescence-based sensor at a bottom of the reaction vessel or suspend the fluorescence-based sensor at a distance above the bottom the reaction vessel.

2. The sensor strip of claim 1, comprising a plurality of fluorescence-based sensors disposed or attached to the first end.

3. The sensor strip of claim 1, comprising:
a first fluorescence-based sensor disposed or attached to the first end, the first fluorescence-based sensor configured to detect a pH level; and
a second fluorescence-based sensor disposed or attached to the first end, the second fluorescence-based sensor configured to detect a dissolved oxygen level.

4. The sensor strip of claim 1, wherein the suspended position of the fluorescence-based sensor at a distance above the bottom the reaction vessel includes positioning the fluorescence-based sensor within media that has been introduced into the reaction vessel.

5. The sensor strip of claim 1, wherein the suspended position of the fluorescence-based sensor at a distance above the bottom the reaction vessel includes positioning the fluorescence-based sensor within media that has been introduced into the reaction vessel so that the fluorescence-based sensor is within a volume of space defined by the media that is on top of cells that are at the bottom of the reaction vessel.

6. A cell culture monitoring kit, comprising:
a sensor strip, comprising:
a strip comprising a first end and a second end;
the first end having a fluorescence-based sensor disposed or attached thereto, the first end being further configured to be inserted into a reaction vessel; and
the second end having a stopper configured to engage with a mouth of the reaction vessel so as to secure the sensor strip to the reaction vessel and to position the fluorescence-based sensor at a bottom of the reaction vessel or suspend the fluorescence-based sensor at a distance above the bottom the reaction vessel; and
at least one of:
the reaction vessel; and
a reader configured to impart light onto the fluorescence-based sensor and receive emitted light therefrom.

7. The kit of claim 6, wherein:
the reader comprises a beam combiner assembly configured to generate an excitation light beam for inducing fluorescence;
the fluorescence-based sensor is configured to generate emitted light due to the induced fluorescence by the excitation light beam; and,
the reader comprises a detector to detect the emitted light;
the fluorescence-based sensor is associated with an expected offset, the expected offset being an expected wavelength of the emitted light corresponding to the fluorescence-based sensor generating the emitted light while in presence of dissolved oxygen; and
the detector is configured to detect a wavelength of emitted light relative to the expected offset.

8. An incubation system, comprising:
an incubator configured to contain a reaction vessel within a controlled environment, the controlled environment including a controlled temperature, pressure, lighting, and humidity; and
a rocker unit configured to rock or oscillate the reaction vessel.

9. The incubation system of claim 8, wherein:
the incubator is configured to contain a plurality of reaction vessels; and
the rocker unit is configured to rock or oscillate the plurality of reaction vessels in unison so that each reaction vessel is rocked at the same angular frequency and angular displacement or individually so that one reaction vessel is able to be rocked at the same or different angular frequency as another reaction vessel and so that one reaction vessel is able to be rocked at the same or different angular displacement as another reaction vessel.

10. The incubation system of claim 7, further comprising a rotary unit configured to rotate the reaction vessel in a circular or precession-like manner.

11. The incubation system of claim 10, wherein:
the incubator is configured to contain a plurality of reaction vessels; and
the rotary unit is configured to rotate the plurality of reaction vessels in unison so that each reaction vessel is rotated at the same rate or individually so that one reaction vessel is able to be rotated at the same or different rate as another reaction vessel.

12. The incubation system of claim 8, comprising:
a sensor strip, comprising:
a strip comprising a first end and a second end;
the first end having a fluorescence-based sensor disposed or attached thereto, the first end being further configured to be inserted into the reaction vessel; and
the second end having a stopper configured to engage with a mouth of the reaction vessel so as to secure the sensor strip to the reaction vessel and to position the fluorescence-based sensor at a bottom of the reaction vessel or suspend the fluorescence-based sensor at a distance above the bottom the reaction vessel; and
a reader configured to impart light onto the sensor and receive emitted light therefrom.

13. An incubation system, comprising:
an incubator configured to contain a reaction vessel within a controlled environment, the controlled environment including a controlled temperature, pressure, lighting, and humidity; and
a gas circulation system configured to introduce and/or remove gas to/from the reaction vessel.

14. The incubation system of claim 13, wherein:
the incubator is configured to contain a plurality of reaction vessels; and
the gas circulation system is configured to introduce and/or remove gas to/from the plurality of reaction vessels in unison so that the gas for each reaction vessel is adjusted at the same rate, concentration, and volume or individually so that the gas for one reaction vessel is able to be adjusted at the same or different rate, concentration, and volume as another reaction vessel.

15. The incubation system of claim 13, further comprising:
a sensor strip, comprising:
a strip comprising a first end and a second end;
the first end having a fluorescence-based sensor disposed or attached thereto, the first end being further configured to be inserted into the reaction vessel; and
the second end having a stopper configured to engage with a mouth of the reaction vessel so as to secure the sensor strip to the reaction vessel and to position the fluorescence-based sensor at a bottom of the reaction vessel or suspend the fluorescence-based sensor at a distance above the bottom the reaction vessel; and
a reader configured to impart light onto the sensor and receive emitted light therefrom.

16. A method of monitoring a cell culture, the method comprising:
inserting a sensor strip into a reaction vessel, the sensor strip comprising a first end having a fluorescence-based sensor disposed or attached thereto, the first end being configured to spearhead the insertion;
allowing the fluorescence-based sensor to be within a volume of space defined by media that has been introduced into the reaction vessel, the media being growth media to support cell growth;
imparting light onto the fluorescence-based sensor and receiving emitted light therefrom.

17. The method of claim 16, wherein the reaction vessel has a living cell culture growing within the reaction vessel prior to inserting the sensor strip.

18. The method of claim 16, further comprising analyzing the emitted light to detect a pH level and/or a dissolved oxygen level.

19. The method of claim 18, further comprising rocking and/or rotating the reaction vessel based on the detected pH level and/or dissolved oxygen level.

20. The method of claim 18, further comprising adjusting gas within the reaction vessel based on the detected pH level and/or dissolved oxygen level.
